(19) United States
(12) Patent Application Publication
(10) Pub. No.: US 2006/0276510 A1
(43) Pub. Date: Dec. 7, 2006

(54) Dihydropyridine compounds and compositions for headaches

(75) Inventors: Susan Abu-Shakra, Malvern, PA (US); Julian A. Gray, Inzlingen (DE); Yoshiharu Yamanishi, Tsukuba-shi (JP); Nobuyuki Mori, Tsukuba-shi (JP)

Correspondence Address: VENABLE LLP, P.O. BOX 34385, WASHINGTON, DC, 20043-9998, US

(73) Assignee: Eisai Co. Ltd., Bunkyo-ku (JP)

(21) Appl. No.: 11/396,621

(22) Filed: Apr. 4, 2006

Related U.S. Application Data
(60) Provisional application No. 60/667,665, filed on Apr. 4, 2005.
(60) Provisional application No. 60/689,519, filed on Jun. 13, 2005.

(51) Int. Cl. A61K 31/444

(52) U.S. Cl. 514/332

(57) ABSTRACT

The invention provides methods for treating and/or preventing headaches by administering to patients therapeutically effective amounts of 1,2-dihydropyridine compounds, and, optionally, cholinesterase inhibitors and/or anti-migraine agents. The headaches may be primary headaches, such as migraines, or secondary headaches. The invention also provides combinations, commercial packages, and pharmaceutical compositions comprising therapeutically effective amounts of 1,2-dihydropyridine compounds and, optionally, cholinesterase inhibitors and/or anti-migraine agents. The 1,2-dihydropyridine compound may be, for example, 3-(2-cyanophenyl)-5-(2-pyridyl)-1-phenyl-1,2-dihydropyridin-2-one. The cholinesterase inhibitor may be, for example, 1-benzyl-4-((5,6-dimethoxy-1-indanon)-2-yl)methylpiperidine.

### RELATED APPLICATIONS

This application claims priority under 35 U.S.C. §119 to U.S. Provisional Application No. 60/689,519 filed Jun. 13, 2005, and to U.S. Provisional Application No. 60/667,665 filed Apr. 4, 2005, the disclosures of which are incorporated by reference herein in their entirety.

### FIELD OF THE INVENTION

The invention provides pharmaceutical compositions comprising dihydropyridine compounds and methods for treating a variety of diseases and disorders using dihydropyridine compounds. The dihydropyridine compounds can optionally be used in conjunction with other drugs, such as cholinesterase inhibitors or anti-migraine agents, for treating a variety of diseases and disorders.

### BACKGROUND OF THE INVENTION

The pathophysiology of migraines has been described in detail in previous articles. Waeber et al, Neurology, 61(Suppl 4):S9-S20 (2003). Although the mechanisms leading to migraines are still mostly unknown, a neurogenic theory of migraine is reported. This theory proposed that trigeminovascular fibers projecting to the meninges are activated during a migraine, leading to neuropeptide release and a neurogenic sterile inflammation in the dura mater. Consequently, a hyperalgesic condition is established.

There is evidence that at least ten receptors (5-HT_{1B}, 5-HT_{1D}, 5-HT_{1F}, 5-HT_{2B}, NK-1, GABA_{A}, N-methyl-D-aspartate (NMDA), α-amino-3-hydroxy-5-methylisoxazole-4-propionic acid (AMPA), class III metabotropic glutamate receptors, opioids μ receptors) represent potential targets for anti-migraine agents. Sumatriptan effects the 5-HT_{1B/1D/1F} receptors. Mitsikostas et al, Brain Research Reviews, 35:20-35 (2001). The role of AMPA receptors, kainate receptors, and mGluR (metabotropic glutamate receptors) in headaches has been studied. It was reported that blocking AMPA, but not kainate receptors, or activating mGluR4 receptors may play a role in the treatment of headaches. Mitsikostas et al, British Journal of Pharmacology, 127:623-630 (1999). LY293558 (an α-amino-3-hydroxy-5-methyl-4-isoxazolepropionic acid (AMPA)/kainate (KA) receptor antagonist) was compared to sumatriptan and placebo in preclinical models for migraines. The efficacy rates for a sustained response at two hours after infusion were 25% (4 patients/16 patients) for placebo; 69% (9 patients/13 patients) for LY293558; and 87% (13 patients/15 patients) for sumatriptan. It was concluded that AMPA/GLuR5 antagonism may provide a target for migraine therapy. Ramadan et al, Cephalalgia, 21:267-272 (2001); Sang et al, Cephalalgia, 24:596-602 (2004).

There is a need in the art for new compounds and new methods of treating diseases and disorders that may be mediated to any extent by NMDA receptors, AMPA receptors and/or kainate receptors. The invention is directed to this, as well as other, important ends.

### SUMMARY OF THE INVENTION

The invention provides methods for treatment and/or prophylaxis of headaches (e.g., migraines) in a patient in need thereof by administering a therapeutically effective amount of at least one 1,2-dihydropyridine compound, and, optionally, a therapeutically effective amount of: (i) at least one cholinesterase inhibitor, (ii) at least one anti-migraine agent, or (iii) at least one cholinesterase inhibitor and at least one anti-migraine agent. The headache may be a primary headache or a secondary headache. In one embodiment, the 1,2-dihydropyridine compound is 3-(2-cyanophenyl)-5-(2-pyridyl)-1-phenyl-1,2-dihydropyridin-2-one. In one embodiment, the cholinesterase inhibitor is donepezil. The combination of the 1,2-dihydropyridine compound and the cholinesterase inhibitor may unexpectedly produce synergistic effects in the treatment and/or prophylaxis of headaches.

In other embodiments, the invention provides pharmaceutical compositions comprising a therapeutically effective amount of at least one 1,2-dihydropyridine compound (e.g., 3-(2-cyanophenyl)-5-(2-pyridyl)-1-phenyl-1,2-dihydropyridin-2-one). The invention provides pharmaceutical compositions comprising a therapeutically effective amount of: (i) at least one cholinesterase inhibitor (e.g., donepezil) and (ii) at least one 1,2-dihydropyridine compound (e.g., 3-(2-cyanophenyl)-5-(2-pyridyl)-1-phenyl-1,2-dihydropyridin-2-one). The invention also provides pharmaceutical compositions comprising a therapeutically effective amount of: (i) at least one anti-migraine agent and (ii) at least one 1,2-dihydropyridine compound (e.g., 3-(2-cyanophenyl)-5-(2-pyridyl)-1-phenyl-1,2-dihydropyridin-2-one). The invention provides pharmaceutical compositions comprising a therapeutically effective amount of: (i) at least one cholinesterase inhibitor (e.g., donepezil); (ii) at least one 1,2-dihydropyridine compound (e.g., 3-(2-cyanophenyl)-5-(2-pyridyl)-1-phenyl-1,2-dihydropyridin-2-one); and (iii) at least one anti-migraine agent.

### BRIEF DESCRIPTION OF THE FIGURES

FIG. 1 shows the effect of Compound A (i.e., 3-(2-cyanophenyl)-5-(2-pyridyl)-1-phenyl-1,2-dihydropyridin-2-one) on withdrawal latency of ipsilateral hind paw.

FIG. 2 shows the effect of Compound A (i.e., 3-(2-cyanophenyl)-5-(2-pyridyl)-1-phenyl-1,2-dihydropyridin-2-one) on withdrawal latency of contralateral hind paw.

### DETAILED DESCRIPTION OF THE INVENTION

“Patient” refers to animals, preferably mammals, more preferably humans. The term “patient” includes men and women; and includes adults, children and neonates. In one embodiment, the patient can be an animal companion, such as a dog or a cat.

“Headaches” refer to primary headaches and secondary headaches according to The International Classification of Headache Disorders as described in Cephalagia, 24(Suppl. 1):9-160 (2004), the disclosure of which is incorporated by reference herein in its entirety. The primary headaches and secondary headaches may be episodic or chronic. “Episodic headaches” refers to patients who experience headaches from 1 to 14 days per month. “Chronic headaches” refers to patients who experience headaches 15 or more days per month. The methods of treatment may be acute or chronic. “Acute treatment” refers to treating a headache on an as-need basis, e.g., upon the onset of the headache a patient is administered a therapeutically effective amount of the compounds or compositions of the invention as described herein. “Chronic treatment” refers to treating a headache on a continual (e.g., daily) basis whether or not the patient is experiencing a headache at the time of administration of the therapeutically effective amount of the compounds or compositions of the invention as described herein. Headaches may cause one or more symptoms such as vertigo, nausea, vomiting, fatigue, aura, photophobia, phonophobia, and the like.

“Primary headaches” include migraines, tension headaches, cluster headaches, paroxysmal hemicrania, short-lasting unilateral neuralgiform headache attacks with conjunctival injection and tearing (SUNCT), trigeminal autonomic cephalalgia, stabbing headaches, cough headaches, exertional headaches, headaches associated with sexual activity, hypnic headaches, thunderclap headaches, hemicrania continua, or a new daily-persistent headache.

“Secondary headaches” include headaches attributed to head and/or neck traumas; headaches attributed to cranial and/or cervical vascular disorders; headaches attributed to non-vascular intracranial disorders; headaches attributed to drugs; headaches attributed to withdrawal from drugs; headaches attributed to infections, headaches attributed to disturbances of homeostasis, headaches or facial pain attributed to disorders of facial structures and/or cranial structures; or headaches attributed to a psychiatric disorders.

“Migraine” refers to a symptom complex occurring periodically and characterized by pain in the head, usually unilateral pain in the head. Migraines generally have a pulsating quality and moderate or severe intensity that inhibits or prohibits daily activities. One or more symptoms caused by migraines include vertigo, nausea, vomiting, fatigue, aura, photophobia, phonophobia, and the like. Migraines may occur with or without aura. Exemplary migraines include classic migraines, common migraines, complicated migraines, menstrual migraines, premenstrual migraines, ophthalmic migraines, ophthalmoplegic migraines, fulgurating migraines, Harris' migraines, and hemiplegic migraines. Neurologic symptoms can occur which are caused by migraines, but which are not followed by head pain. For example, abdominal pain and vomiting can occur without head pain as the sole expression of a migraine.

“Administered separately” with reference to the administration of two or more compounds to treat and/or prevent the diseases and disorders described herein includes, for example, the sequential administration of the compounds in any order or the simultaneous administration of the compounds. Simultaneous administration of the compounds means that the compounds are administered to the patient at substantially the same time or at exactly the same time, depending on the mode of administration. The sequential administration of the compounds may occur in any order and may occur with any amount of time elapsing between administration of the compounds. Sequential administration may be based on factors that would influence which of the compounds should be administered first and which should be administered second, and how much time should elapse between administration of the compounds. For example, when two or more compounds are administered separately and sequentially, factors that effect when the compounds are administered to the patient include, for example, (a) the time(s) that provides the best efficacy for the compound being administered, (b) the time(s) that provides the fewest side effects for the compound being administered, (c) the dosage of the compound, (d) the route of administration of the compound, (e) the disease or disorder being treated, (f) the patient being treated, (g) the in vivo relationship of the compounds being administered, and other such factors known in the art. Preferably, the time intervals for sequential administration are chosen so that the effect on the disease or disorder being treated in the combined use of the active ingredients is greater than additive when compared to the effect which would be obtained by use of only one of the active ingredients.

The term “combination” refers to the 1,2-dihydropyridine compound and the second active ingredient (e.g., cholinesterase inhibitors) being administered separately as distinct pharmaceutical compositions or formulations (e.g., a first pharmaceutical composition comprising a 1,2-dihydropyridine compound and a second pharmaceutical composition comprising a cholinesterase inhibitor). The pharmaceutical compositions or formulations can have the same or different modes of administration.

“Active ingredient” refers to the 1,2-dihydropyridines, cholinesterase inhibitors, anti-migraine agents, and other compounds described herein that are responsible for treatment and/or prophylaxis of a disease or disorder.

“Monotherapy” is a therapy which uses only one active ingredient for treatment and/or prophylaxis of a disease or disorder.

“Combination therapy” is a therapy where two or more active ingredients are administered separately or are administered in the form of a pharmaceutical composition for the treatment and/or prophylaxis of a disease.

“Therapeutically effective amount” refers to the amount of the active ingredient that is necessary for the treatment and/or prophylaxis of a disease. When two or more active ingredients are administered for combination therapy, the term “therapeutically effective amount” refers to the amount of active ingredients that are necessary for treatment and/or prophylaxis of a disease and includes, for example: (a) a therapeutically effective amount of a first active ingredient and a therapeutically effective amount of a second active ingredient (i.e., the amount of each active ingredient that would be used for monotherapy for the treatment and/or prophylaxis of a disease is used for the combination therapy); (b) a therapeutically effective amount of a first active ingredient and a sub-therapeutic amount of a second active ingredient, which in combination effectively provide for treatment and/or prophylaxis of a disease (e.g., the sub-therapeutic amount of the second active ingredient can be used in combination therapy to achieve a result that would be equal to or greater than the result that the second active ingredient would achieve if it was used for monotherapy); (b) a sub-therapeutic amount of a first active ingredient and a therapeutically effective amount of a second active ingredient, which in combination effectively provide for treatment and/or prophylaxis of a disease (e.g., the sub-therapeutic amount of the first active ingredient can be used in combination therapy to achieve a result that would be equal to or greater than the result that the first active ingredient would achieve if it was used for monotherapy); and (d) a sub-therapeutic amount of a first active ingredient and a sub-therapeutic amount of a second active ingredient, which in combination therapy provide for treatment and/or prophylaxis of a disease or disorder (e.g., the sub-therapeutic amount of the first active ingredient can be used in combination therapy to achieve a result that would be equal to or greater than the result that the first active ingredient would achieve if it was used for monotherapy; and the sub-therapeutic amount of the second active ingredient can be used in combination therapy to achieve a result that would be equal to or greater than the result that the second active ingredient would achieve if it was used for monotherapy). The same therapeutic/sub-therapeutic amounts can be used when there are three or more active ingredients used in combination therapy. For example, (a) there may be therapeutically effective amounts of all three active ingredients; (b) there may be therapeutically effective amounts of two active ingredients and a sub-therapeutic amount of a third active ingredient; (c) there may be a therapeutically effective amount of one active ingredient and sub-therapeutic amounts of two other active ingredients; or (d) there may be sub-therapeutic amounts of all three active ingredients.

“Commercial packages,” also known as kits, can include a combination of (i) a first pharmaceutical composition or formulation comprising the 1,2-dihydropyridine compound; (ii) a second pharmaceutical composition or formulation comprising the second active ingredient (e.g., cholinesterase inhibitors); (iii) instructions approved by the FDA for using the pharmaceutical compositions or formulations for treating or preventing the disease; and (iv) optionally other materials to administer the pharmaceutical compositions or formulations (e.g., syringes, diluents, medical gloves, hand sanitizers, and the like); to monitor drug levels in the body; to support patient compliance with medication dosing; or to monitor the status of the disease. The commercial package can supply enough medication and materials for days, weeks or months. In another embodiment, “commercial packages” can include (i) pharmaceutical composition or formulation comprising both the 1,2-dihydropyridine compound and the second active ingredient (e.g., cholinesterase inhibitors); (ii) instructions approved by the FDA for using the pharmaceutical composition or formulation for treating or preventing the disease; and (iii) optionally other materials to administer the pharmaceutical compositions or formulations (e.g., syringes, diluents, medical gloves, hand sanitizers, and the like); to monitor drug levels in the body; to support patient compliance with medication dosing; or to monitor the status of the disease. The commercial package can supply enough medication and materials for days, weeks or months.

“Hydrate” refers to a compound containing a molecule of water of crystallization. The molecule of water of crystallization can be an integer of 1 or more, such as 1 to 10; or can be any fraction greater than 0 or a fraction of an integer from 1 to 10. For example, the hydrate may be represented as compound.¼H₂O; compound.½H₂O; compound. ¾H₂O; compound.2H₂O; compound.5½H₂O; compound.6H₂O; and the like. The “compound” can be any described herein, such as 3-(2-cyanophenyl)-5-(2-pyridyl)-1-phenyl-1,2-dihydropyridin-2-one.

“Pharmaceutically acceptable salts” are well known in the art and include those of inorganic acids, such as hydrochloride, sulfate, hydrobromide and phosphate; and those of organic acids, such as formate, acetate, trifluoroacetate, methanesulfonate, benzenesulfonate and toluenesulfonate. When certain substituents are selected, the compounds of the invention can form, for example, alkali metal salts, such as sodium or potassium salts; alkaline earth metal salts, such as calcium or magnesium salts; organic amine salts, such as a salt with trimethyl-amine, triethylamine, pyridine, picoline, dicyclohexylamine or N,N′-dibenzylethylenediamine. One skilled in the art will recognize that the compounds of the invention can be made in the form of any other pharmaceutically acceptable salt.

In one embodiment, the compounds used in the methods and compositions described herein are 1,2-dihydropyridine compounds. The 1,2-dihydropyridine compound may be any known in the art. The term “1,2-dihydropyridine compound” includes 1,2-dihydropyridine compounds, pharmaceutically acceptable salts of 1,2-dihydropyridine compounds, stereoisomers of 1,2-dihydropyridine compounds, pharmaceutically acceptable salts of stereoisomers of 1,2-dihydropyridine compounds, hydrates of 1,2-dihydropyridine compounds, hydrates of pharmaceutically acceptable salts of 1,2-dihydropyridine compounds, stereoisomers of hydrates of 1,2-dihydropyridine compounds, and stereoisomer of hydrates of pharmaceutically acceptable salts of 1,2-dihydropyridine compounds.

Preferably, the 1,2-dihydropyridine compound used in the methods and compositions described herein is a compound of Formula (I): 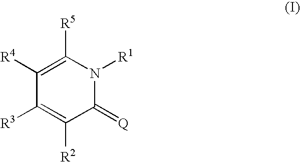
wherein

Q is NH, O or S;

R¹, R², R³, R⁴ and R⁵ are each independently hydrogen, halogen, C₁₋₆ alkyl, or —X-A;

X is a single bond, an optionally substituted C₁₋₆ alkylene, an optionally substituted C₂₋₆ alkenylene, an optionally substituted C₂₋₆ alkynylene, —O—, —S—, —CO—, —SO—, —SO₂—, —N(R⁶)—, —N(R⁷)—CO—, —CO—N(R⁸)—, —N(R⁹)—CH₂—, —CH₂—N(R¹⁰)—, —CH₂—CO—, —CO—CH₂—, —N(R¹¹)—S(O)ₘ—, —S(O)ₙ—N(R¹²)—, —CH₂—S(O)ₚ—, —S(O)_{q—CH}₂—, —CH₂—O—, —O—CH₂—, —N(R¹³)—CO—N(R¹⁴)— or —N(R¹⁵)—CS—N(R¹⁶)—;

R⁶, R⁷, R⁸, R⁹, R¹⁰, R¹¹, R¹², R¹³, R¹⁴, R¹⁵ and R¹⁶ are each independently hydrogen C₁₋₆ alkyl, or C₁₋₆ alkoxy;

m, n, p and q are each independently an integer of 0, 1 or 2;

A is an optionally substituted C₃₋₈ cycloalkyl, an optionally substituted C₃₋₈ cycloalkenyl, an optionally substituted 5- to 14-membered non-aromatic heterocyclic ring, an optionally substituted C₆₋₁₄ aromatic hydrocarbocyclic ring, or an optionally substituted 5- to 14-membered aromatic heterocyclic ring; provided that 3 groups among R¹, R², R³, R⁴ and R⁵ are —X-A; and that the residual 2 groups among R¹, R², R³, R⁴ and R⁵ are independently hydrogen, halogen, or C₁₋₆ alkyl.

In one embodiment, the following compounds are excluded from the scope of the compound of Formula (I): (1) when Q is O; R¹ and R⁵ are hydrogen; and R², R³ and R⁴ are phenyl; (2) when Q is O; R¹ and R⁴ are hydrogen; and R², R³ and R⁵ are phenyl; and (3) when Q is O; R¹ and R² are hydrogen; and R³, R⁴ and R⁵ are phenyl.

In another embodiment preferred embodiment, the 1,2-dihydropyridine compound used in the methods and compositions described herein is a compound of Formula (II):

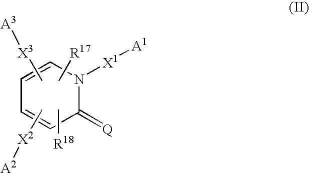

wherein

Q is NH, O or S;

X¹, X² and X³ are each independently a single bond, an optionally substituted C₁₋₆ alkylene, an optionally substituted C₂₋₆ alkenylene, an optionally substituted C₂₋₆ alkynylene, —O—, —S—, —CO—, —SO—, —SO₂—, —N(R⁶)—, —N(R⁷)—CO—, —CO—N(R⁸)—, —N(R⁹)—CH₂—, —CH₂—N(R¹⁰)—, —CH₂—CO—, —CO—CH₂—, —N(R¹¹)—S(O)ₘ—, —S(O)ₙ—N(R¹²)—, —CH₂—S(O)ₚ—, —S(O)_{q}—CH₂—, —CH₂—O—, —O—CH₂—, —N(R¹³)—CO—N(R¹⁴)— or —N(R¹⁵)—CS—N(R¹⁶);

R⁶, R⁷, R⁸, R⁹, R¹⁰, R¹¹, R¹², R¹³, R¹⁴, R¹⁵ and R¹⁶ are each independently hydrogen C₁₋₆ alkyl, or C₁₋₆ alkoxy;

m, n, p and q are each independently an integer of 0, 1 or 2;

A¹, A² and A³ are each independently an optionally substituted C₃₋₈ cycloalkyl, an optionally substituted C₃₋₈ cycloalkenyl, an optionally substituted 5- to 14-membered non-aromatic heterocyclic ring, an optionally substituted C₆₋₁₄ aromatic hydrocarbocyclic ring, or an optionally substituted 5 to 14-membered aromatic heterocyclic ring; and

R¹⁷ and R¹⁸ are each independently hydrogen, halogen, or C₁₋₆ alkyl.

In another embodiment, the invention provides the compound of Formula (II) wherein X¹, X² and X³ are each independently a single bond, an optionally substituted C₁₋₆ alkylene, an optionally substituted C₂₋₆ alkenylene, or an optionally substituted C₂₋₆ alkynylene. The substituents may be one or more of —O—, —S—, —CO—, —SO—, —SO₂—, —N(R⁶)—, —N(R⁷)—CO—, —CO—N(R⁸)—, —N(R⁹)—CH₂—, —CH₂—N(R¹⁰)—, —CH₂—CO—, —CO—CH₂—, —N(R¹¹)—S(O)ₘ—, —S(O)ₙ—N(R¹²)—, —CH₂—S(O)ₚ—, —S(O)_{q}—CH₂—, —CH₂—O—, —O—CH₂—, —N(R¹³)—CO—N(R¹⁴)— and —N(R¹⁵)—CS—N(R¹⁶)—;

R⁶, R⁷, R⁸, R⁹, R¹⁰, R¹¹, R¹², R¹³, R¹⁴, R¹⁵ and R¹⁶ are each independently hydrogen, C₁₋₆ alkyl, or C₁₋₆ alkoxy;

m, n, p and q are each independently an integer of 0, 1 or 2;

A¹, A² and A³ are each independently an optionally substituted C₃₋₈ cycloalkyl, an optionally substituted C₃₋₈ cycloalkenyl, an optionally substituted 5- to 14-membered non-aromatic heterocyclic ring, an optionally substituted C₆₋₁₄ aromatic hydrocarbocyclic ring, or an optionally substituted 5- to 14-membered aromatic heterocyclic ring.

The substituents for the 1,2-dihydropyridine compounds of the invention may be one or more of hydroxy; halogen; nitrile; nitro; C₁₋₆ alkyl; C₂₋₆ alkenyl; C₂₋₆ alkynyl [wherein the alkyl, alkenyl, and alkynyl can independently and optionally be substituted with one or more groups selected from hydroxy, nitrile, halogen, C₁₋₆ alkylamino, di(C₁₋₆ alkyl) amino, C₂₋₆ alkenylamino, di(C₂₋₆ alkenyl)amino, C₂₋₆ alkynylamino, di(C₂₋₆ alkynyl)amino, N—C₁₋₆ alkyl-N—C₂₋₆ alkenylamino, N—C₁₋₆ alkyl-N—C₂₋₆ alkynylamino, N—C₂₋₆ alkenyl-N—C₂₋₆alkynylamino, aralkyloxy, TBDMS oxy, C₁₋₆ alkylsulfonylamino, C₁₋₆ alkylcarbonyloxy, C₂₋₆ alkenylcarbonyloxy, C₂₋₆ alkynylcarbonyloxy, N—C₁₋₆ alkylcarbamoyl, N—C₂₋₆ alkenylcarbamoyl, and N—C₁₋₆ alkynylcarbamoyl]; C₁₋₆ alkoxy; C₂₋₆ alkenyloxy; C₂₋₆ alkynyloxy [wherein the alkoxy, alkenyloxy, and alkynyloxy may independently and optionally be substituted with one or more groups selected from C₁₋₆ alkylamino, aralkyloxy, and hydroxy]; C₁₋₆ alkylthio; C₂₋₆ alkenylthio; C₂₋₆ alkynylthio [wherein the alkylthio, alkenylthio, and alkynylthio may independently and optionally be substituted with one or more groups selected from hydroxy, nitrile, halogen, C₁₋₆ alkylamino, aralkyloxy, TBDMS oxy, C₁₋₆ alkylsulfonylamino, C₁₋₆ alkylcarbonyloxy, and C₁₋₆ alkylcarbamoyl]; optionally substituted carbonyl [which may be substituted with C₁₋₆ alkoxy, amino, C₁₋₆ alkylamino, di(C₁₋₆ alkyl)amino, C₂₋₆ alkenylamino, di(C₂₋₆ alkenyl)amino, C₂₋₆ alkynylamino, di(C₂₋₆ alkynyl)amino, N—C₁₋₆alkyl-N—C₂₋₆ alkenylamino, N—C₁₋₆ alkyl-N—C₂₋₆ alkynylamino and N—C₂₋₆ alkenyl-N—C₂₋₆ alkynylamino]; an optionally substituted amino [which may be substituted with one or two groups selected from C₁₋₆ alkyl, C₂₋₆ alkenyl, C₂₋₆ alkynyl, C₁₋₆ alkylsulfonyl, C₂₋₆ alkenylsulfonyl, C₂₋₆ alkynylsulfonyl, C₁₋₆ alkylcarbonyl, C₂₋₆ alkenylcarbonyl and C₂₋₆ alkynylcarbonyl]; C₁₋₆ alkylsulfonyl; C₂₋₆ alkenylsulfonyl; C₂₋₆ alkynylsulfonyl; C₁₋₆ alkylsulfinyl; C₂₋₆ alkenylsulfinyl C₂₋₆ alkynylsulfinyl; formyl; optionally substituted C₃₋₈ cycloalkyl; an optionally substituted C₃₋₈ cycloalkenyl [where the cycloalkyl group and/or the cycloalkenyl group may independently and optionally be substituted with one or more groups selected from hydroxy, halogen, nitrile, C₁₋₆ alkyl, C₁₋₆ alkyloxy, C₁₋₆ alkyloxy C₁₋₆ alkyl, and aralkyl]; a 5- to 14-membered non-aromatic heterocyclic ring [which may optionally be substituted with one or more groups selected from hydroxy, halogen, nitrile, C₁₋₆ alkyl, C₁₋₆ alkyloxy, C₁₋₆ alkyloxy C₁₋₆ alkyl, and aralkyl]; C₆₋₁₄ aromatic hydrocarbocyclic ring [which may optionally be substituted with one or more groups selected from hydroxy, halogen, nitrile, C₁₋₆ alkyl, C₁₋₆ alkyloxy, C₁₋₆ alkyloxy C₁₋₆ alkyl, and aralkyl]; and a 5- to 14-membered aromatic heterocyclic ring [which may optionally be substituted with one or more groups selected from hydroxy, halogen, nitrile, C₁₋₆ alkyl, C₁₋₆ alkyloxy, C₁₋₆ alkyloxy C₁₋₆ alkyl, and aralkyl].

In another embodiment, the invention provides compounds of Formula (II) wherein A¹, A² and A³ are each independently an optionally substituted C₃₋₈ cycloalkyl, an optionally substituted C₃₋₈ cycloalkenyl or an optionally substituted 5- to 14-membered non-aromatic hetero ring. In another embodiment, the invention provides the compound of Formula (II) wherein A¹, A² and A³ are each independently an optionally substituted C₆₋₁₄ aromatic hydrocarbon ring or an optionally substituted 5- to 14-membered aromatic hetero ring. In another embodiment, the invention provides the compound of Formula (II) wherein A¹, A² and A³ are each independently phenyl, pyrrolyl, pyridyl, pyridazinyl, pyrimidinyl, pyrazinyl, thienyl, thiazolyl, furyl, naphthyl, quinolyl, iso-quinolyl, indolyl, benzimidazolyl, benzothiazolyl, benzoxazolyl, imidazopyridyl, carbazolyl, cyclopentyl, cyclohexyl, cyclohexenyl, dioxinyl, adamantyl, pyrrolidinyl, piperidinyl, piperazinyl or morpholyl; any of which may optionally have substituents. In another embodiment, the invention provides the compound of Formula (II) wherein A¹, A² and A³ are each independently selected from: 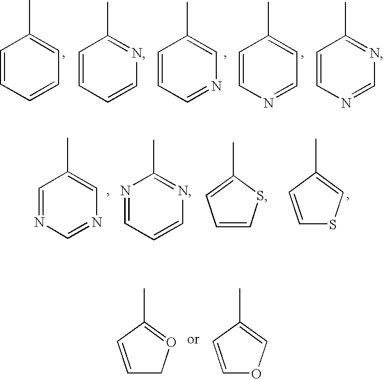
each of which may optionally be substituted. In another embodiment, the invention provides the compound of Formula (II) wherein A¹, A² and A³ are each independently substituted with hydroxyl, halogen, amino, or nitrile. In another embodiment, the invention provides the compound of Formula (II) wherein A¹, A² and A³ are each independently hydroxyl, halogen, amino, nitrile, or nitro. In another embodiment, the invention provides the compound of Formula (II) wherein Q is oxygen.

In another embodiment, the invention provides the compounds of Formula (I) or (II) wherein X¹, X² and X³ are each independently a single bond, —CH₂—, —CH(OH)—, —CH₂—CH₂—, —CH═CH—, —C≡C—, —O— or —CO—. In another embodiment, the invention provides the compounds of Formula (I) or (II) wherein X¹, X² and X³ are each a single bond. In another embodiment, the invention provides the compounds of Formula (I) or (II) wherein R¹⁷ and R¹⁸ are each independently hydrogen, fluorine, chlorine, bromine, iodine, methyl, ethyl, n-propyl, or iso-propyl. In another embodiment, the invention provides the compounds of Formula (I) or (II) wherein R¹⁷ and R¹⁸ are each hydrogen.

With respect to the 1,2-dihydropyridine compounds of the invention, the halogen atom indicates fluorine, chlorine, bromine, iodine and the like, and the preferable atoms include fluorine, chlorine and bromine.

With respect to the 1,2-dihydropyridine compounds of the invention, the C₁₋₆ alkyl indicates an alkyl having 1 to 6 carbons, and examples include linear chain or branched chain alkyl groups such as methyl, ethyl, n-propyl, iso-propyl, n-butyl, iso-butyl, sec-butyl (1-methylpropyl), tert-butyl, iso-pentyl, n-pentyl, tert-pentyl (1,1-dimethylpropyl), 1,2-dimethylpropyl, 2,2-dimethylpropyl (neopentyl), 1-ethylpropyl, 2-methylbutyl, n-hexyl, iso-hexyl, 1,2-dimethylbutyl, 1-ethyl-1-methylpropyl, 1-ethyl-2-methylpropyl, 1,1,2-trimethylpropyl, 1,2,2-trimethylpropyl, 1-ethylbutyl, 1-methylbutyl, 1,1-dimethylbutyl, 2,2-dimethylbutyl, 1,3-dimethylbutyl, 2,3-dimethylbutyl, 3,3-dimethylbutyl, 2-ethylbutyl, 1-methylpentyl, 2-methylpentyl, 3-methylpentyl, and the like.

With respect to the 1,2-dihydropyridine compounds of the invention, the C₂₋₆ alkenyl indicates an alkenyl group having 2 to 6 carbons, and examples include vinyl, 1-ethylethenyl (1-buten-2-yl), allyl (2-propenyl), 1-propenyl, iso-propenyl, 2-methyl-1-propenyl, 1-methyl-1-propenyl (2-buten-2-yl), 2-methyl-2-propenyl, 1-methyl-2-propenyl, 1-butenyl (1-buten-1-yl), 2-butenyl (2-buten-1-yl), 3-butenyl, 1-pentenyl, 1-hexenyl, 1,3-hexadienyl, 1,6-hexadienyl, and the like.

With respect to the 1,2-dihydropyridine compounds of the invention, the C₂₋₆ alkynyl indicates an alkynyl group having 2 to 6 carbons, and examples include ethynyl, 1-propynyl, 2-propynyl, 1-butynyl, 2-butynyl, 3-butynyl, 1-ethyl-1-propynyl, 1-ethynyl-2-propynyl, 2-methyl 3-butenyl, 1-pentynyl, 1-hexynyl, 1,3-hexadiynyl, 1,6-hexadiynyl, and the like.

With respect to the 1,2-dihydropyridine compounds of the invention, the C₁₋₆ alkoxy indicates an alkoxy group having 1 to 6 carbons, and examples include methoxy, ethoxy, n-propoxy, iso-propoxy, n-butoxy, iso-butoxy, sec-butoxy, tert-butoxy, n-pentyloxy, iso-pentyloxy, tert-pentyloxy, 1,2-dimethylpropoxy, neopentyloxy, 1-ethylpropoxy, 1-methylbutoxy, 2-methylbutyoxy, n-hexyloxy, iso-hexyloxy, 1-ethyl-1-methylpropoxy, 1-ethyl-2-methylpropoxy, 1,1,2-trimethylpropoxy, 1,2,2-trimethylpropoxy, 1,1-dimethylbutoxy, 2,2-dimethylbutoxy, 2,3-dimethylbutoxy, 3,3-dimethylbutoxy, 2-ethylbutoxy, 1,3-dimethylbutoxy, 1-ethylbutoxy, 1-methylbutoxy, 1-methylpentyloxy, 2-methylpentyloxy, 3-methylpentyloxy, and the like.

With respect to the 1,2-dihydropyridine compounds of the invention, the C₂₋₆ alkynyloxy indicates an alkynyloxy group having 2 to 6 carbon atoms, and examples include ethynyloxy, 1-propynyloxy, 2-propynyloxy, 1-butynyloxy, 2-butynyloxy, 3-butynyloxy, 1-methyl-2-propynyloxy, 1-ethyl-2-propynyloxy, 1-ethynyl-2-propynyloxy, 1-pentynyloxy, 1-hexynyloxy, 1,3-hexadiynyloxy, 1,6-hexadiynyloxy, and the like.

With respect to the 1,2-dihydropyridine compounds of the invention, the C₂₋₆ alkenyloxy indicates an alkenyloxy group having 2 to 6 carbons, and examples include vinyloxy, 1-ethylethenyloxy (1-buten-2-yloxy), allyloxy (2-propenyloxy), 1-propenyloxy, iso-propenyloxy, 2-methyl-1-propenyloxy, 1-methyl-1-propenyloxy (2-buten-2-yloxy), 2-methyl-2-propenyloxy, 1-methyl-2-propenyloxy (1-buten-3-yloxy), 1-butenyloxy (1-buten-1-yloxy), 2-butenyloxy (2-buten-1-yloxy), 3-butenyloxy, 1-pentenyloxy, 1-hexenyloxy, 1,3-hexadienyloxy, 1,6-hexadienyloxy, and the like.

With respect to the 1,2-dihydropyridine compounds of the invention, the C₃₋₈ cycloalkyl indicates a cycloalkyl group composed of 3 to 8 carbon atoms, and examples include cyclopropyl, cyclobutyl, cyclopentyl, cyclohexyl, cycloheptyl, cyclooctyl, and the like.

With respect to the 1,2-dihydropyridine compounds of the invention, the C₃₋₈ cycloalkenyl indicates a cycloalkenyl group composed of 3 to 8 carbon atoms, and examples include cyclopropen-1-yl, 2-cyclopropen-1-yl, cyclobuten-1-yl, 2-cyclobuten-1-yl, 1,3-cyclobutadien-1-yl, cyclopenten-1-yl, 2-cyclopenten-1-yl, 3-cyclopenten-1-yl, 1,3-cyclopentadien-1-yl, 1,4-cyclopentadien-1-yl, 2,4-cyclopentadien-1-yl, cyclohexen-1-yl, 2-cyclohexen-1-yl, 3-cyclohexen-1-yl, 1,3-cyclohexadien-1-yl, 1,4-cyclohexadien-1-yl, 1,5-cyclohexadien-1-yl, 2,4-cyclohexadien-1-yl, 2,5-cyclohexadien-1-yl, cyclohepten-1-yl, 2-cyclohepten-1-yl, 3-cyclohepten-1-yl, 4-cyclohepten-1-yl, 1,3-cyclopentadien-1-yl, 1,4-cyclopentadien-1-yl, 1,5-cycloheptadien-1-yl, 1,6-cycloheptadien-1-yl, 2,4-cycloheptadien-1-yl, 2,5-cycloheptadien-1-yl, 2,6-cycloheptadien-1-yl, 3,5-cycloheptadien-1-yl, 1,3,5-cycloheptatrien-1-yl, 1,3,6-cycloheptatrien-1-yl, 1,4,6-cycloheptatrien-1-yl, 2,4,6-cycloheptatrien-1-yl, cycloocten-1-yl, 2-cycloocten-1-yl, 3-cycloocten-1-yl, 4-cycloocten-1-yl, 1,3-cyclooctadien-1-yl, 1,4-cyclooctadien-1-yl, 1,5-cyclooctadien-1-yl, 1,6-cyclooctadien-1-yl, 1,7-cyclooctadien-1-yl, 2,4-cyclooctadien-1-yl, 2,5-cyclooctadien-1-yl, 2,6-cyclooctadien-1-yl, 2,7-cyclooctadien-1-yl, 3,5-cyclooctadien-1-yl, 3,6-cyclooctadien-1-yl, 1,3,5-cyclooctatrien-1-yl, 1,3,6-cyclooctatrien-1-yl, 1,3,7-cyclooctatrien-1-yl, 1,4,6-cyclooctatrien-1-yl, 1,4,7-cyclooctatrien-1-yl, 1,5,7-cyclooctatrien-1-yl, 2,4,6-cyclooctatrien-1-yl, 2,4,7-cyclooctatrien-1-yl group, and the like.

With respect to the 1,2-dihydropyridine compounds of the invention, the 5- to 14-membered non-aromatic heterocyclic ring means a mono-cyclic, di-cyclic, or tri-cyclic 5- to 14-membered non-aromatic heterocyclic ring which contains one or more hetero atoms selected from nitrogen, sulfur, and oxygen. Specific examples include pyrrolidinyl, pyrrolinyl, piperidyl, piperazinyl, pyrazolidinyl, imidazolidinyl, morpholinyl, tetrahydrofuryl, tetrahydropyranyl, dihydrofuryl, dihydropyranyl, imidazolinyl, oxazolinyl, and the like. Further, a group derived from a pyridone ring and a non-aromatic condensed ring (for example, a group derived from a phthalimide ring, a succinimide ring, and the like) are also included in the non-aromatic heterocyclic ring.

With respect to the 1,2-dihydropyridine compounds of the invention, the C₆₋₁₄ aromatic hydrocarbocyclic ring and the aryl mean an aromatic hydrocarbocyclic ring which is composed of 6 to 14 carbon atoms, a mono-cyclic ring, and a condensed di-cyclic, tri-cyclic and the like. Specific examples include phenyl, indenyl, 1-naphthyl, 2-naphthyl, azulenyl, heptalenyl, biphenyl, indathenyl, acenaphthyl, fluorenyl, phenalenyl, phenanthrenyl, anthracenyl, cyclopentacyclooctenyl, benzocyclooctenyl and the like.

With respect to the 1,2-dihydropyridine compounds of the invention, the 5- to 14-membered aromatic heterocyclic ring and the heteroaryl ring mean mono-cyclic, di-cyclic, or tri-cyclic 5- to 14-membered aromatic heterocyclic ring which contain one or more hetero atoms selected from nitrogen, sulfur, and oxygen. Specific examples include (1) aromatic heterocyclic rings containing nitrogen such as pyrrolyl, pyridyl, pyridazinyl, pyrimidinyl, pyrazinyl, triazolyl, tetrazolyl, benzotriazolyl, pyrazolyl, imidazolyl, benzimidazolyl, indolyl, iso-indolyl, indolizinyl, prenyl, indazolyl, quinolyl, iso-quinolyl, quinoliziyl, phthalazyl, naphthylidinyl, quinoxalyl, quinazolinyl, cynnolinyl, pteridinyl, imidazotriazinyl, pyrazinopyridazinyl, acridinyl, phenanthridinyl, carbazolyl, carbazolinyl, perimidinyl, phenanthrolinyl, phenazinyl, imidazopyridyl, imidazopyrimidinyl, or pyrazolopyridyl; (2) aromatic heterocyclic rings containing sulfur such as thienyl or benzothienyl; (3) aromatic heterocyclic rings containing oxygen such as furyl, pyranyl, cyclopentapyranyl, benzofuryl, or iso-benzofuryl; and (4) aromatic heterocyclic rings containing 2 or more different hetero atoms such as thiazolyl, iso-thiazolyl, benzothiazolyl, benzothiadiazolyl, phenothiazinyl, isoxazolyl, furazanyl, phenoxazinyl, oxazolyl, isoxazoyl, benzoxazolyl, oxadiazolyl, pyrazoloxadiazolyl, imidazothiazolyl, thienofuranyl, furopyrrolyl or pyridoxadinyl.

In another embodiment, the 1,2-dihydropyridine compound used in the methods and compositions described herein is preferably a compound of Formula (III): 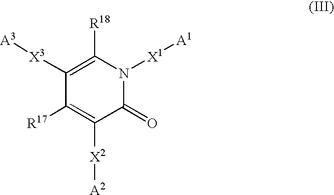
wherein X¹, X², X³, A¹, A², A³, R¹⁷ and R¹⁸ have the same meanings as defined in the above compound of Formula (II).

In another embodiment, the invention provides the compounds of Formula (III) wherein A¹, A² and A³ are each independently an optionally substituted C₆₋₁₄ aromatic hydrocarbon ring or 5- to 14-membered aromatic hetero ring. In another embodiment, the invention provides the compounds of Formula (III) wherein A¹, A² and A³ are each independently phenyl, pyrrolyl, pyridyl, pyridazinyl, pyrimidinyl, pyrazinyl, thienyl, thiazolyl, furyl, naphthyl, quinolyl, iso-quinolyl, indolyl, benzimidazolyl, benzothiazolyl, benzoxazolyl, imidazopyridyl, carbazolyl, cyclopentyl, cyclohexyl, cyclohexenyl, dioxinyl, adamantyl, pyrrolidinyl, piperidinyl, piperazinyl, or morpholyl; wherein each may optionally be substituted. In another embodiment, the invention provides the compounds of Formula (III) wherein A¹, A² and A³ are each independently selected from: 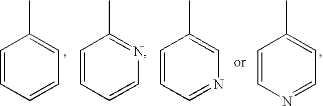
each of which may optionally be substituted. In another embodiment, the invention provides the compounds of Formula (III) wherein the bonding site of the substituent at A¹, A² and A³ are in the a-position of the carbon atom bonding to the group X¹, X² and X³, respectively. In another embodiment, the invention provides the compounds of Formula (III) wherein X¹, X² and X³ are single bonds. In another embodiment, the invention provides the compounds of Formula (III) wherein R⁷ and R¹⁸ are hydrogen.

In one embodiment, the 1,2-dihydropyridine compound used in the methods and compositions described herein is preferably Compound A: 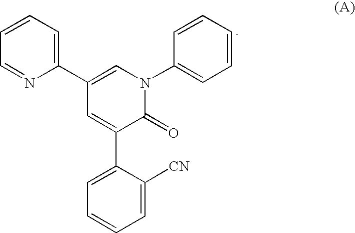
The IUPAC name for Compound A is 2-(2-oxo-1-phenyl-5-pyridin-2-yl-1,2-dihydropyridin-3-yl)benzonitrile. Compound A may also be referred to as 3-(2-cyanophenyl)-5-(2-pyridyl)-1-phenyl-1,2-dihydropyridin-2-one.

Throughout the specification, the terms “Compound A,” “2-(2-oxo-1-phenyl-5-pyridin-2-yl-1,2-dihydropyridin-3-yl)benzonitrile,” and “3-(2-cyanophenyl)-5-(2-pyridyl)-1-phenyl-1,2-dihydropyridin-2-one” are intended to include pharmaceutically acceptable salts thereof, stereoisomers thereof, pharmaceutically acceptable salts of stereoisomers thereof, hydrates thereof, hydrates of pharmaceutically acceptable salts thereof, stereoisomers of hydrates thereof, and stereoisomer of hydrates of pharmaceutically acceptable salts thereof. In another embodiment, the terms “Compound A,” “2-(2-oxo-1-phenyl-5-pyridin-2-yl-1,2-dihydropyridin-3-yl)benzonitrile,” and “3-(2-cyanophenyl)-5-(2-pyridyl)-1-phenyl-1,2-dihydropyridin-2-one” are intended to include pharmaceutically acceptable salts thereof, hydrates thereof, and hydrates of pharmaceutically acceptable salts thereof.

In other embodiments, the 1,2-dihydropyridine compounds that are useful in the methods and compositions of the invention are 3-(2-cyanophenyl)-5-(2-methylsulfonylaminophenyl)-1-phenyl-1,2-dihydropyridin-2-one; 3-(2-chloro-3-pyridyl)-5-(2-pyridyl)-1-phenyl-1,2-dihydropyridin-2-one; 3-(2-cyanophenyl)-5-(2-pyridyl)-1-phenyl-1,2-dihydropyridin-2-one; 3-(2-cyanophenyl)-5-(2-pyridyl)-1-(3-nitrophenyl)-1,2-dihydropyridin-2-one; 3-(2-cyanophenyl)-5-(2-pyridyl)-1-(3-aminophenyl)-1,2-dihydropyridin-2-one; 3-(2-cyanophenyl)-5-(2-pyridyl)-1-(3-methylsulfonylaminophenyl)-1,2-dihydropyridin-2-one; 3-(2-cyanophenyl)-5-(2-pyridyl)-1-(3-methylaminophenyl)-1,2-dihydropyridin-2-one; 3-(2-cyanophenyl)-5-(2-pyridyl)-1-(3-dimethylaminophenyl)-1,2-dihydropyridin-2-one; 3-(2-cyanophenyl)-5-(2-pyridyl)-1-[3-(5-methoxymethyl-2-oxazolidinon-3-yl)-phenyl]-1,2-dihydropyridin-2-one; 3-(2-cyanophenyl)-5-(2-pyridyl)-1-(3-methoxycarbonylphenyl)-1,2-dihydropyridin-2-one; 3-(2-cyanophenyl)-5-(2-pyridyl)-1-(3-methylaminocarbonylphenyl)-1,2-dihydropyridin-2-one; 3-(2-cyano-3-pyridyl)-5-(2-pyridyl)-1-phenyl-1,2-dihydropyridin-2-one; 3-(2-chlorophenyl)-5-(2-pyridyl)-1-(4-hydroxyphenyl)-1,2-dihydropyridin-2-one; 3-(2-chlorophenyl)-5-(2-pyridyl)-1-(4-dimethylaminoethoxyphenyl)-1,2-dihydropyridin-2-one; 3-(2-cyanophenyl)-5-(2-pyridyl)-1-(3-formylphenyl)-1,2-dihydropyridin-2-one; 3-(2-cyanophenyl)-5-(2-pyridyl)-1-(3-hydroxymethylphenyl)-1,2-dihydropyridin-2-one; 3-(2-cyanophenyl)-5-(2-pyridyl)-1-(3-cyanomethylphenyl)-1,2-dihydropyridine-2-one; 3-(2-cyanophenyl)-5-(2-pyridyl)-1-(3-acetylaminomethylphenyl)-1,2-dihydropyridin-2-one; 3-(2-cyanophenyl)-5-(2-pyridyl)-1-(3-methylsulfonylaminomethylphenyl)-1,2-dihydropyridin-2-one; 3-(2-cyanophenyl)-5-(2-pyridyl)-1-(3-acetoxymethylphenyl)-1,2-dihydropyridin-2-one; 3-(2-cyanophenyl)-5-(2-pyridyl)-1(4-methylthiophenyl)-1,2-dihydropyridin-2-one; 3-(2-cyanophenyl)-5-(2-pyridyl)-1-(4-methylsulfonylpheny-1)-1,2-dihydropyridin-2-one; 3-(2-cyanophenyl)-5-(2-formylthiophen-3-yl)-1-phenyl-1,2-dihydropyridin-2-one; 3-(2-cyanophenyl)-5-(2-diethylaminomethylthiophen-3-yl)-1-phenyl-1,2-dihydropyridin-2-one; 3-(2-cyanophenyl)-5-(2-hydroxymethylthiophen-3-yl)-phenyl-1,2-dihydropyridine-2-one; 3-(2-cyanophenyl)-5-(2-pyridyl)-1-benzyl-1,2-dihydropyridin-2-one; 3-(2-cyanophenyl)-5-(2-pyridyl)-1-(3-pyridyl)-1,2-dihydropyridin-2-one; 3-(2-cyanophenyl)-5-phenyl-(2-pyridyl)-1,2-dihydropyridin-2-one; 3-(2-cyanophenyl)-1,5-diphenyl-1,2-dihydropyridin-2-one; 3-(2-cyanophenyl)-5-(2-methoxyphenyl)-1-phenyl-1,2-dihydropyridin-2-one; 3-(2-cyanophenyl)-5-(3,4-dimethoxyphenyl)-1-phenyl-1,2-dihydropyridin-2-one; 3-(2-cyanophenyl)-5-(thiophen-3-yl)-1-phenyl-1,2-dihydropyridin-2-one; 3-(2-cyanophenyl)-5-(2-fluorophenyl)-1-phenyl-1,2-dihydropyridin-2-one; 3-(2-cyanophenyl)-5-(thiophen-2-yl)-1-phenyl-1,2-dihydropyridin-2-one; 3-(2-cyanophenyl)-5-(3-furfuryl)-1-phenyl-1,2-dihydropyridin-2-one; 3-(2-cyanophenyl)-5-(2-furfuryl)-1-phenyl-1,2-dihydropyridin-2-one; 3-(2-chlorophenyl)-5-(2-pyridyl)-1-phenyl-1,2-dihydropyridin-2-one; 3-(2-methoxycarbonylphenyl)-5-(2-pyridyl)-1-phenyl-1,2-dihydropyridin-2-one; 3-phenyl-5-(2-pyridyl)-1-phenyl-1,2-dihydropyridin-2-one; 3-(2-fluorophenyl)-5-(2-pyridyl)-1-phenyl-1,2-dihydropyridin-2-one; 3-(2-chlorophenyl)-5-(2-pyridyl)-1-(3-methoxyphenyl)-1,2-dihydropyridin-2-one; 3-(2-fluoro-3-pyridyl)-5-(2-pyridyl)-1-phenyl-1,2-dihydropyridin-2-one; 3-(4-methoxy-3-pyridyl)-5-(2-pyridyl)-1-phenyl-1,2-dihydropyridin-2-one; 3-(2-fluoro-3-pyridyl)-5-(2-pyridyl)-1-(3-methoxyphenyl)-1,2-dihydropyridin-2-one; 3-(2-fluoro-3-pyridyl)-5-(2-pyridyl)-1-(3-fluorophenyl)-1,2-dihydropyridin-2-one; 3-(2-cyanophenyl)-5-(2-pyridyl)-1-(4-fluorophenyl)-1,2-dihydropyridin-2-one; 3-(2-cyanophenyl)-5-(2-pyridyl)-1-(3-fluorophenyl)-1,2-dihydropyridin-2-one; 3-(2-cyanophenyl)-5-(2-pyridyl)-1-(4-methoxyphenyl)-1,2-dihydropyridin-2-one; 3-(2-cyanophenyl)-5-(2-pyridyl)-1-(3-methoxy-phenyl)-1,2-dihydropyridin-2-one; 3-phenyl-5-(2-pyridyl)-1-(3-fluorophenyl-)-1,2-dihydropyridin-2-one; 3-(2-chlorophenyl)-5-(2-pyridyl)-1-(4-fluorophenyl)-1,2-dihydropyridin-2-one; 3-(2-cyanophenyl)-5-(2-pyridyl)-1-(4-formylphenyl)-1,2-dihydropyridin-2-one; 3-(2-cyanophenyl)-5-(2-pyridyl)-1-(2-formylphenyl)-1,2-dihydropyridin-2-one; 3-(2-cyanophenyl)-5-(2-pyridyl)-1-(3-chlorophenyl)-1,2-dihydropyridin-2-one; 3-(2-cyanophenyl)-5-(2-pyridyl)-1-(3-tolyl)-1,2-dihydropyridin-2-one; 3-(2-cyanophenyl)-5-(2-pyridyl)-1-(3-trifluoromethylphenyl)-1,2-dihydropyridin-2-one; 3-(2-cyanophenyl)-5-(2-pyridyl)-1-(thiophen-3-yl)-1,2-dihydropyridin-2-one; 3-(2-cyanophenyl)-5-(2-pyridyl)-1-(3-furfuryl)-1,2-dihydropyridin-2-one; 3-(2-cyanophenyl)-5-(2-pyridyl)-1-(4-tolyl)-1,2-dihydropyridin-2-one; 3-(2-cyanophenyl)-5-(2-pyridyl)-1-(4-trifluoromethylphenyl)-1,2-dihydropyridin-2-one; 3-(2-cyanophenyl)-5-(2-pyridyl)-1-(2-methoxypyridin-5-yl)-1,2-dihydropyri-din-2-one; 3-(2-cyanophenyl)-5-(2-pyridyl)-1-(pyrimidin-5-yl)-1,2-dihydrop-yridin-2-one; 3-(2-cyanophenyl)-5-(2-pyridyl)-1-(3-benzyloxymethylpyridin-5-yl)-1,2-dihydropyridin-2-one; 3-(2-cyanophenyl)-5-(2-pyridyl)-1-(2-ethylthiopyridin-5-yl)-1,2-dihydropyridin-2-one; 3-(2-cyanophenyl)-5-(2-pyridyl-)-1-(4-pyridyl)-1,2-dihydropyridin-2-one; 3-(2-cyanophenyl)-5-(2-pyridyl)-1-(3-methoxypyridin-5-yl)-1,2-dihydropyridin-2-one; 3-(2-cyanophenyl)-5-(2-pyridyl)-1-(2-chloropyridin-5-yl)-1,2-dihydropyridin-2-one; 3-(2-cyanophenyl)-5-(2-pyridyl)-1-(2-fluoropyridin-5-yl)-1,2-dihydropyridin-2-one; 3-(2-cyanophenyl)-5-(2-pyridyl)-1-(2-methoxyphenyl)-1,2-dihydropyridin-2-one; 3-phenyl-5-(2-pyridyl)-1-(3-pyridyl)-1,2-dihydropyridin-2-one; 3-(2-chlorophenyl)-5-(2-pyridyl)-1-(3-pyridyl)-1,2-dihydropyridin-2-one; 3-(thiophen-3-yl)-5-(2-pyridyl)-1-(3-pyridyl)-1,2-dihydropyridin-2-one; 3-(2,6-dimethylphenyl)-5-(2-pyridyl)-1-(3-pyridyl)-1,2-dihydropyridin-2-one; 3-(2-cyanothiophen-3-yl)-5-(2-pyridyl)-1-(3-pyridyl)-1,2-dihydropyridin-2-one; 3-(2-fluoro-3-pyridyl)-5-(2-pyridyl)-1-(3-pyridyl)-1,2-dihydropyridin-2-one; 3-(2-chlorophenyl)-5-(2-pyridyl)-1-(3-hydroxyphenyl)-1-,2-dihydropyridin-2-one; 3-(2-chlorophenyl)-5-(2-pyridyl)-1-(3-dimethylaminoethoxyphenyl)-1,2-dihydropyridin-2-one; 3-(2-chlorophenyl)-5-(2-pyridyl)-1-(3-dimethylaminopropoxyphenyl)-1,2-dihydropyridin-2-one; 3-(2-cyanophenyl)-5-(2-pyridyl)-1-(2-hydroxymethylphenyl)-1,2-dihydropyridin-2-one; 3-(2-cyanophenyl)-5-(2-pyridyl)-1-(4-cyanomethylphenyl)-1,2-dihydropyridin-2-one; 3-(2-cyanophenyl)-5-(2-pyridyl)-1-(2-cyanomethylphenyl)-1,2-dihydropyridin-2-one; 3-(2-cyanophenyl)-5-(6-diethylaminomethyl-2-pyridyl)-1-phenyl-1,2-dihydropyridin-2-one; 3-(2-cyanophenyl)-1-phenyl-5-(2-pyrimidinyl)-1,2-dihydropyridin-2-one; 3-(2-hydroxypyridin-6-yl)-1-phenyl-5-(2-pyridyl)-1,2-dihydropyridin-2-one; 1-(2-aminobenzothiazol-6-yl)-3-(2-cyanophenyl)-5-(2-pyridyl)-1,2-dihydropyridin-2-one; 3-(2-cyanophenyl)-5-(2-pyridyl)-1-(1-benzyl-1,2,3,6-tetrahydropyridin-5-yl)-1,2-dihydropyridin-2-one; 3-[2-(5-methyl-1,2,4-oxadiazol-3-yl)phenyl]-1-phenyl-5-(2-pyridyl)-1,2-dihydropyridin-2-one; 3-(2-cyanophenyl)-5-(6-methylpyridin-2-yl)-1-phenyl-1,2-dihydropyridin-2-one; 3-(2-cyanophenyl)-5-(5-methylpyridin-2-yl)-1-phenyl-1,2-dihydropyridin-2-one; 3-(2-cyanophenyl)-5-(3-hydroxypyridin-2-yl)-1-phenyl-1,2-dihydropyridin-2-one; 3-(2-cyanophenyl)-1-phenyl-5-(2-thiazolyl)-1,2-dihydropyridin-2-one; 3-(2-cyanophenyl)-5-(2-methoxypyridin-6-yl)-1-phenyl-1,2-dihydropyridin-2-one; 1-(4-aminophenyl)-3-(2-cyanophenyl)-5-(2-pyridyl)-1,2-dihydropyridin-2-one; 1-(3-aminophenyl)-3-(2-cyanophenyl)-5-(2-pyrimidinyl)-1,2-dihydropyridin-2-one; 3-(2-cyanophenyl)-5-(2-pyridyl)-1-(2-aminotoluen-4-yl)-1-,2-dihydropyridin-2-one; 3-(2-cyanophenyl)-1-[3-(dimethylaminoethoxy)phenyl]-5-(2-pyridyl)-1,2-dihydropyridin-2-one; 3-(2-cyanophenyl)-1-[3-(piperidinoethoxy)phenyl]-5-(2-pyridyl)-1,2-dihydropyridin-2-one; 3-(2-cyanophenyl)-1-[3-(pyrrolidinoethoxy)phenyl]-5-(2-pyridyl)-1,2-dihyd-ropyridin-2-one; 3-(2-cyanophenyl)-1-[3-(diisoproylaminoethoxy)phenyl]-5-(-2-pyridyl)-1,2-dihydropyridin-2-one; 3-(2-cyanophenyl)-1-[3-(4-piperidinobutyl-1-oxy)phenyl]-5(2-pyridyl)-1,2-dihydropyridin-2-one; 3-(2-cyanophenyl)-1-(4-nitrophenyl)-5-(2-pyridyl)-1,2-dihydropyridin-2-one; 1-phenyl-5-(2-pyridyl)-3-(2-thiazolyl)-1,2-dihydropyridin-2-one; 3 -(2-cyanophenyl)-1-(3-pyridyl)-5-(2-pyrimidinyl)-1,2-dihydropyridin-2-one; 3-(2-fluoropyridin-3-yl)-1-phenyl-5-(2-pyrimidinyl)-1,2-dihydropyridin-2-one; 3-(2-cyanopyridin-3-yl)-1-phenyl-5-(2-pyrimidinyl)-1,2-dihydropyridin-2-one; 3-(2-cyanophenyl)-1-(3-nitrophenyl)-5-(2-pyrimidinyl)-1,2-dihydropyridin-2-one; 3-(2-nitrophenyl)-1-phenyl-5-(2-pyridyl)-1,2-dihydropyridin-2-one; 3-(2-formylthiophen-3-yl)-5-(2-pyridyl)-1-phenyl-1,2-dihydropyrid-in-2-one; 3-(2-cyanophenyl)-5-(2-pyridyl)-1-(2-naphthyl)-1,2-dihydropyridin-2-one; 3-(2-cyanophenyl)-5-(2-pyridyl)-1-(1-naphthyl)-1,2-dihydropyridin-2-one; 5-(2-aminopyridin-6-yl)-3-(2-cyanophenyl)-1-phenyl-1,2-dihydropyridin-2-one; 5-(2-bromopyridin-6-yl)-3-(2-cyanophenyl)-1-phenyl-1,2-dihydropyridin-2-one; 3-(2-cyanophenyl)-5-(2-morphorinopyridin-6-yl)-1-phenyl-1,2-dihydropyridin-2-one; 3-(2-cyanophenyl)-1-(3-hydoxyphenyl)-5-(2-pyridyl)-1,2-dihydropyridin-2-one; 3-(2-cyanophenyl)-1-[3-(4-piperidyloxy)]phenyl-5-(2-pyridyl)-1,2-dihydropyridin-2-one; 1-[3-(N-acetylpiperidin-4-yl-oxy)phenyl]-3-(2-cyanophenyl)-5-(2-pyridyl)-1,2-dihydropyridin-2-one; 3-(2-cyanophenyl)-1-{3-[1-(methanesufonyl)piperidin-4-yl-oxy]phenyl}-5-(2-pyridyl)-1,2-dihydropyridin-2-one; 1-[3-(N-methylpiperidin-4-yl-oxy)pheny-1]-3-(2-cyanophenyl)-5-(2-pyridyl)-1,2-dihydropyridin-2-one; 3-(6-chloro-1H-benzimidazol-2-yl)-5-(2-pyridyl)-1-phenyl-1,2-dihydropyridin-2-one; 3-(2-cyanophenyl)-5-(2-pyridyl)-1-(2-nitrotoluen-4-yl)-1,2-dihydropyridin-2-one; 3-(2-cyanothiophen-3-yl)-5-(2-pyridyl)-1-phenyl-1,2-dihydropyridin-2-one; 3-[2-(5-oxazolyl)phenyl]-1-phenyl-5-(2-pyridyl)-1,2-dihydropyridin-2-one; 3-[2-(5-oxazolyl)thiophen-3-yl]-1-phenyl-5-(2-pyridyl)-1,2-dihydropyridin-2-one; and 3-(2-ethoxycarbonylvinylthiophen-3-yl)-5-(2-pyridyl)-1-phenyl-1,2-dihydropyridin-2-one.

The 1,2-dihydropyridine compounds and methods for making the 1,2-dihydropyridine compounds are described in U.S. Pat. No. 6,949,571, US Publication No. 2004/0023973, and PCT Publication No. WO 03/047577, WO 04/009553, WO 06/004100, and WO 06/004107, the disclosures of which are incorporated by reference herein in their entirety.

In one embodiment, the compounds used in the methods and compositions described herein are cholinesterase inhibitors. The cholinesterase inhibitors may be any known in the art. The term “cholinesterase inhibitor” includes cholinesterase inhibitors, pharmaceutically acceptable salts of cholinesterase inhibitors, stereoisomers of cholinesterase inhibitors, and pharmaceutically acceptable salts of stereoisomers of cholinesterase inhibitors. Exemplary cholinesterase inhibitors include donepezil, tacrine, physostigmine, pyridostigmine, neostigmine, rivastigmine, galantamine, citicoline, velnacrine, huperzine (e.g., huperzine A), metrifonate, heptastigmine, edrophonium, phenserine, tolserine, phenethylnorcymserine, quilostigmine, ganstigmine, epastigmine, upreazine, 3-[1-(phenylmethyl)-4-piperidinyl]-1-(2,3,4,5-tetrahydro-1H-1-benzazepin-8-yl)-1-propanone, (2-[2-(1-benzylpiperidin-4-yl)ethyl]-2,3-dihydro-9-methoxy-1H-pyrrolo [3,4-b]quinolin-1-one), and the like. In one embodiment, the cholinesterase inhibitor is donepezil, tacrine, galantamine, or rivastigmine.

In one embodiment, the preferred cholinesterase inhibitors used in the methods and compositions described herein are compounds of formula (IV): 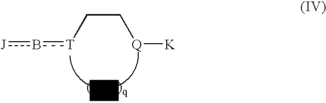
wherein J is (a) a substituted or unsubstituted group selected from (i) phenyl, (ii) pyridyl, (iii) pyrazyl, (iv) quinolyl, (v) cyclohexyl, (vi) quinoxalyl, and (vii) furyl; (b) a monovalent or divalent group, in which the phenyl can have one or more substituents selected from (i) indanyl, (ii) indanonyl, (iii) indenyl, (iv) indenonyl, (v) indanedionyl, (vi) tetralonyl, (vii) benzosuberonyl, (viii) indanolyl, and (ix) C₆H₅—CO—CH(CH₃)—; (c) a monovalent group derived from a cyclic amide compound; (d) a lower alkyl; or (e) R²¹—CH═CH—, in which R²¹ is hydrogen or a lower alkoxycarbonyl; B is —(CHR²²)ᵣ—, —CO—(CHR²²)ᵣ—, —NR⁴—(CHR²²)ᵣ—, —CO—NR⁵—(CHR²²)ᵣ—, —CH═CH—(CHR²²)ᵣ—, —OCOO—(CHR²²)ᵣ—, —OOC—NH—(CHR²²)ᵣ—, NH—CO—(CHR²²)ᵣ—, —CH₂—CO—NH—(CHR²²)ᵣ—, —(CH₂)₂—NH—(CHR²²)ᵣ—, —CH(OH)—(CHR²²)ᵣ—, ═(CH—CH═CH)_{b}—, ═CH—(CH₂)_{c}—, ═(CH—CH)_{d}═, —CO—CH═CH—CH₂—, —CO—CH₂—CH(OH)—CH₂—, —CH(CH₃)—CO—NH—CH₂—, —CH═CH═CO—NH—(CH₂)₂—, —NH—, —O—, —S—, a dialkylaminoalkyl-carbonyl or a lower alkoxycarbonyl; R⁴ is hydrogen, lower alkyl, acyl, lower alkylsulfonyl, phenyl, substituted phenyl, benzyl, or substituted benzyl; R⁵ is hydrogen, lower alkyl or phenyl; r is zero or an integer of 1 to 10; R²² is hydrogen or methyl so that one alkylene group can have no methyl branch or one or more methyl branches; b is an integer of 1 to 3; c is zero or an integer of 1 to 9; d is zero or an integer of 1 to 5; T is nitrogen or carbon; Q is nitrogen, carbon or 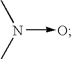
q is an integer of 1 to 3; K is hydrogen, phenyl, substituted phenyl, arylalkyl in which the phenyl can have a substituent, cinnamyl, a lower alkyl, pyridylmethyl, cycloalkylalkyl, adamantanemethyl, furylmethyl, cycloalkyl, lower alkoxycarbonyl or an acyl; and [symbol] is a single bond or a double bond.

In the compound of formula (IV), J is preferably (a) or (b), more preferably (b). In the definition of (b), a monovalent group (2), (3) and (5) and a divalent group (2) are preferred. The group (b) preferably includes the formulae shown below: 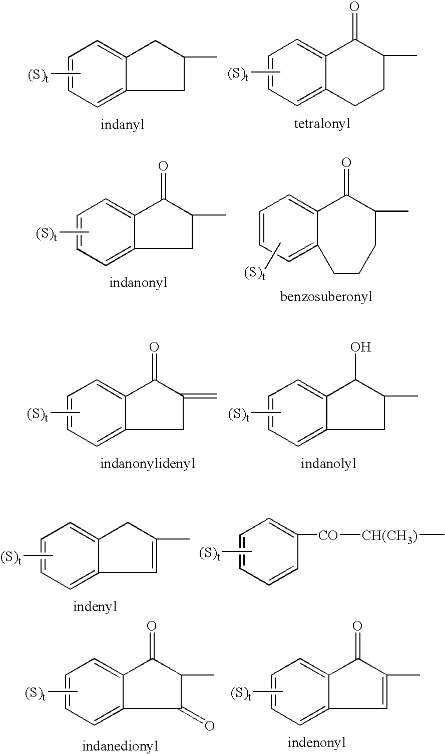
wherein t is an integer of 1 to 4; and each S is independently hydrogen or a substituent, such as a lower alkyl having 1 to 6 carbon atoms or a lower alkoxy having 1 to 6 carbon atoms. Among the substituents, methoxy is most preferred. The phenyl is most preferred to have 1 to 3 methoxy thereon. (S)ₜ can form methylene dioxy or ethylene dioxy on two adjacent carbon atoms of the phenyl. Of the above groups, indanonyl, indanedionyl and indenyl, optionally having substituents on the phenyl, are the most preferred.

In the definition of B, —(CHR²²)ᵣ—, —CO—(CHR²²)ᵣ—, ═(CH—CH═CH)_{b}—, ═CH—(CH₂)_{c}— and ═(CH—CH)_{d}═ (where b is an integer of 1 to 3; c is zero or an integer of 1 to 9; d is zero or an integer of 1 to 5) are preferable. The group —(CHR²²)ᵣ— in which R²² is hydrogen and r is an integer of 1 to 3, and the group ═CH—(CH₂)_{c}— are most preferable. The preferable groups of B can be connected with (b) of J, in particular (b)(2). The ring containing T and Q in formula (I) can be 5-, 6- or 7-membered. It is preferred that Q is nitrogen, T is carbon or nitrogen, and q is 2; or that Q is nitrogen, T is carbon, and q is 1 or 3; or that Q is carbon, T is nitrogen and q is 2. It is preferable that K is a phenyl, arylalkyl, cinnamyl, phenylalkyl or a phenylalkyl having a substituent(s) on the phenyl.

In one embodiment, the preferred cholinesterase inhibitors used in the methods and compositions described herein are compounds of formula (V): 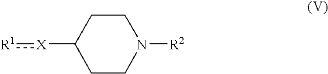
wherein R¹ is a (1) substituted or unsubstituted phenyl; (2) a substituted or unsubstituted pyridyl; (3) a substituted or unsubstituted pyrazyl; (4) a substituted or unsubstituted quinolyl; (5) a substituted or unsubstituted indanyl; (6) a substituted or unsubstituted cyclohexyl; (7) a substituted or unsubstituted quinoxalyl; (8) a substituted or unsubstituted furyl; (9) a monovalent or divalent group derived from an indanone having a substituted or unsubstituted phenyl; (10) a monovalent group derived from a cyclic amide compound; (11) a lower alkyl; or (12) R³—CH═C—, where R³ is a hydrogen atom or a lower alkoxycarbonyl; X is —(CH₂)ₙ—, —C(O)—(CH₂)ₙ—, —N(R⁴)—(CH₂)ₙ—, —C(O)—N(R⁵)—(CH₂)ₙ—, —CH═CH—(CH₂)ₙ—, —O—C(O)—O—(CH₂)ₙ—, —O—C(O)—NH—(CH₂)ₙ—, —CH═CH—CH═CO—, —NH—C(O)—(CH₂)ₙ—, —CH₂—C(O)—NH—(CH₂)ₙ—, —(CH₂)₂—C(O)—NH—(CH₂)ₙ—, —CH(OH)—(CH₂)ₙ—, —C(O)—CH═CH—CH₂—, —C(O)—CH₂—CH(OH)—CH₂—, —CH(CH₃)—C(O)—NH—CH₂—, —CH═CH—C(O)—NH—(CH₂)₂—, a dialkylaminoalkylcarbonyl, a lower alkoxycarbonyl; n is an integer of 0 to 6; R⁴ is hydrogen, lower alkyl, acyl, lower alkylsulfonyl, a substituted or unsubstituted phenyl, or a substituted or unsubstituted benzyl; and R⁵ is hydrogen, lower alkyl, or phenyl; R² is a substituted or unsubstituted phenyl; a substituted or unsubstituted arylalkyl; a cinnamyl; lower alkyl; pyridylmethyl; cycloalkylalkyl; adamantanemethyl; or furoylmethyl; and [symbol] is a single bond or a double bond.

With respect to the cholinesterase inhibitors described herein, the term “lower alkyl” means a straight or branched alkyl having 1 to 6 carbon atoms. Exemplary “lower alkyl” groups include methyl, ethyl, propyl, isopropyl, butyl, isobutyl, sec-butyl, tert-butyl, pentyl (amyl), isopentyl, neopentyl, tert-pentyl, 1-methylbutyl, 2-methylbutyl, 1,2-dimethylpropyl, hexyl, isohexyl, 1-methylpentyl, 2-methyl-pentyl, 3-methylpentyl, 1,1-dimethylbutyl, 1,2-dimethylbutyl, 2,2-dimethylbutyl, 1,3-dimethyl-butyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, and the like. The lower alkyl is preferably methyl, ethyl, propyl or isopropyl; more preferably methyl.

With respect to the cholinesterase inhibitors described herein, specific examples of the substituents for the substituted or unsubstituted phenyl, pyridyl, pyrazyl, quinolyl, indanyl, cyclohexyl, quinoxalyl and furyl in the definition of R¹ include lower alkyl groups having 1 to 6 carbon atoms, such as methyl, ethyl, n-propyl, isopropyl, n-butyl, isobutyl, and tert-butyl groups; lower alkoxy corresponding to the above-described lower alkyl, such as methoxy and ethoxy groups; nitro; halogen, such as chlorine, fluorine and bromine; carboxyl; lower alkoxycarbonyl corresponding to the above-described lower alkoxy, such as methoxycarbonyl, ethoxycarbonyl, isopropoxycarbonyl, n-propoxycarbonyl, and n-butyloxycarbonyl groups; amino; lower monoalkylamino; lower dialkylamino; carbamoyl; acylamino derived from aliphatic saturated monocarboxylic acids having 1 to 6 carbon atoms, such as acetylamino, propionylamino, butyrylamino, isobutyrylamino, valerylamino, and pivaloylamino; cycloalkyloxycarbonyl, such as a cyclohexyloxycarbonyl; lower alkylaminocarbonyl, such as methylaminocarbonyl and ethylaminocarbonyl; lower alkylcarbonyloxy corresponding to the above-defined lower alkyl, such as methylcarbonyloxy, ethylcarbonyloxy, and n-propylcarbonyloxy; halogenated lower alkyl, such as trifluoromethyl; hydroxyl; formyl; and lower alkoxy lower alkyl, such as ethoxymethyl, methoxymethyl and methoxyethyl. The “lower alkyl” and “lower alkoxyl” in the above description of the substituents include all the groups derived from the above-mentioned groups. There may be 1, 2 or 3 substituents, which may be the same or different.

With respect to the cholinesterase inhibitors described herein, when the substituent is phenyl, the following is within the scope of the substituted phenyl: 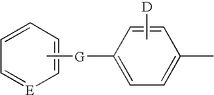
wherein G is —C(O)—, —O—C(O)—, —O—, —CH₂—NH—C(O)—, —CH₂—O—, —CH₂—SO₂—, —CH(OH)—, or —CH₂—S(→O)—; E is carbon or nitrogen; and D is a substituent.

With respect to the cholinesterase inhibitors described herein, preferred examples of the substituents (i.e., “D”) for the phenyl include lower alkyl, lower alkoxy, nitro, halogenated lower alkyl, lower alkoxycarbonyl, formyl, hydroxyl, and lower alkoxy lower alkyl, halogen, and benzyol and benzylsulfonyl. There may be 2 or more substituents, which may be the same or different. Preferred examples of the substituent for the pyridyl include lower alkyl and amino and halogen. Preferred examples of the substituent for the pyrazyl include lower alkoxycarbonyl, carboxyl, acylamino, carbamoyl, and cycloalkyloxycarbonyl.

With respect to the cholinesterase inhibitors described herein, with respect to R¹, the pyridyl is preferably a 2-pyridyl, 3-pyridyl, or 4-pyridyl; the pyrazyl is preferably a 2-pyrazinyl; the quinolyl is preferably a 2-quinolyl or 3-quinolyl; the quinoxalinyl is preferably a 2-quinoxalinyl or 3-quinoxalinyl; and the furyl is preferably a 2-furyl.

With respect to the cholinesterase inhibitors described herein, examples of monovalent or divalent groups derived from an indanone having an unsubstituted or substituted phenyl of (A) or (B): 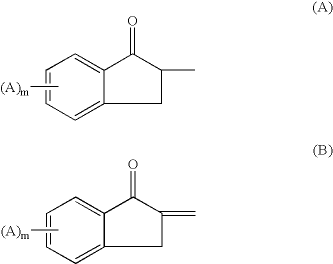
where m is an integer of from 1 to 4, and each A is independently hydrogen, lower alkyl, lower alkoxy, nitro, halogen, carboxyl, lower alkoxycarbonyl, amino, lower monoalkylamino, lower dialkylamino, carbamoyl, acylamino derived from aliphatic saturated monocarboxylic acids having 1 to 6 carbon atoms, cycloalkyloxycarbonyl, lower alkylaminocarbonyl, lower alkylcarbonyloxy, halogenated lower alkyl, hydroxyl, formyl, or lower alkoxy lower alkyl; preferably hydrogen, lower alkyl, or lower alkoxy; most preferably the indanone is unsubstituted or substituted with 1 to 3 methoxy.

With respect to the cholinesterase inhibitors described herein, examples of the monovalent group derived from a cyclic amide compound include quinazolone, tetrahydroisoquinolinone, tetrahydrobenzodiazepinone, and hexahydrobenzazocinone. The monovalent group can be any one having a cyclic amide in the structural formula thereof, and is not limited to the above-described specific examples. The cyclic amide can be one derived from a monocyclic or condensed heterocyclic ring. The condensed heterocyclic ring is preferably one formed by condensation with phenyl. In this case, phenyl can be substituted with a lower alkyl group having 1 to 6 carbon atoms, preferably methyl, or lower alkoxy having 1 to 6 carbon atoms, preferably methoxy.

With respect to the cholinesterase inhibitors described herein, examples of the monovalent group include the following: 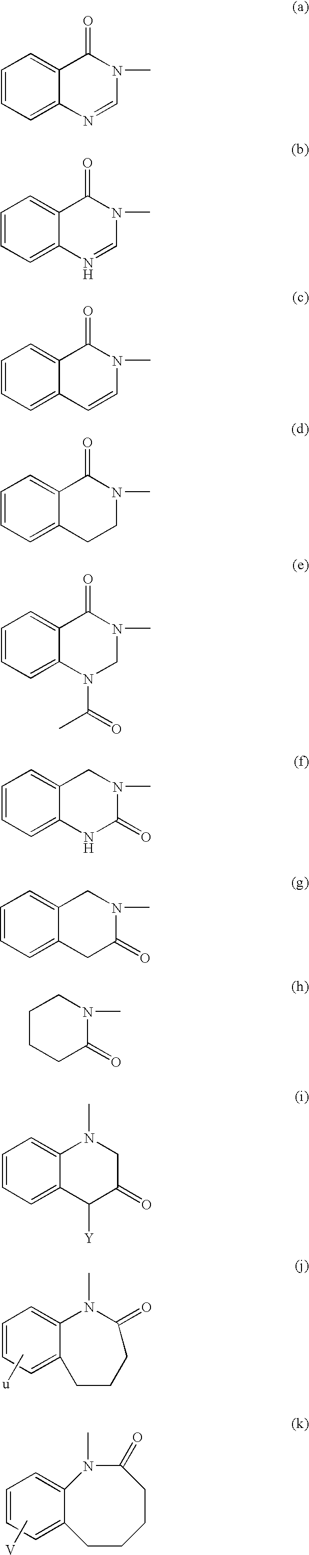 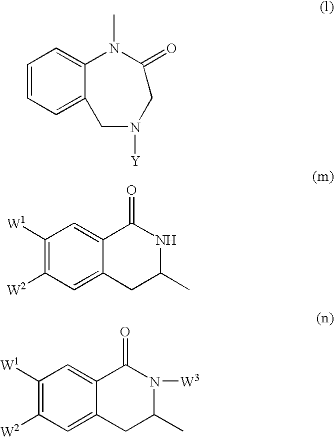
In the above formulae, Y is hydrogen or lower alkyl; V and U are each hydrogen or lower alkoxy (preferably dimethoxy); W¹ and W² are each hydrogen, lower alkyl, or lower alkoxy; and W³ is hydrogen or a lower alkyl. The right hand ring in formulae (j) and (l) is a 7-membered ring, while the right hand ring in formula (k) is an 8-membered ring.

With respect to the cholinesterase inhibitors described herein, the most preferred examples of R¹ include a monovalent group derived from an indanone having an unsubstituted or substituted phenyl and a monovalent group derived from a cyclic amide compound. With respect to the cholinesterase inhibitors described herein, the most preferred examples of X include —(CH₂)ₙ—, amide, or groups represented by the above formulae where n is 2. Thus, it is most preferred that any portion of a group represented by R¹ 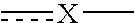
have a carbonyl or amide.

With respect to the cholinesterase inhibitors described herein, the substituents involved in the expressions “a substituted or unsubstituted phenyl” and “a substituted or unsubstituted arylalkyl” in the above definition of R² are the same substituents as those described for the above definitions of phenyl, pyridyl, pyrazyl, quinolyl, indanyl, cyclohexyl, quinoxalyl or furyl in the definition of R¹. The term “arylalkyl” is intended to mean an unsubstituted benzyl or phenethyl or the like. Specific examples of the pyridylmethyl include 2-pyridylmethyl, 3-pyridylmethyl, and 4-pyridylmethyl. Preferred examples of R² include benzyl and phenethyl. The symbol [symbol] means a double or single bond. The bond is a double bond only when R¹ is the divalent group (B) derived from an indanone having an unsubstituted or substituted phenyl, while it is single bond in other cases.

In one embodiment, the preferred cholinesterase inhibitors used in the methods and compositions described herein are compounds of formula (VI): 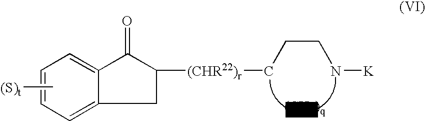
wherein r is an integer of 1 to 10; each R²² is independently hydrogen or methyl; K is a phenalkyl or a phenalkyl having a substituent on the phenyl; each S is independently hydrogen, C₁₋₆ lower alkyl, or C₁₋₆ lower alkoxy; t is an integer of 1 to 4; q is an integer of 1 to 3; with the proviso that (S)ₜ can be methylenedioxy or ethylenedioxy joined to two adjacent carbon atoms of the phenyl.

In other embodiments, the compound of formula (VI) is 1-benzyl-4-((5,6-dimethoxy-1-indanon)-2-yl)methylpiperidine; 1-benzyl-4-((5,6-dimethoxy-1-indanon)-2-ylidenyl)methyl-piperidine; 1-benzyl-4-((5-methoxy-1-indanon)-2-yl)methylpiperidine; 1-benzyl-4-((5,6-diethoxy-1-indanon)-2-yl)methylpiperidine; 1-benzyl-4-((5,6-methnylenedioxy-1-indanon)-2-yl)methylpiperidine; 1-(m-nitrobenzyl)-4-((5,6-dimethoxy-1-indanon)-2-yl)methylpiperidine; 1-cyclohexylmethyl-4-((5,6-dimethoxy-1-indanon)-2-yl)methylpiperidine; 1-(m-fluorobenzyl)-4-((5,6-dimethoxy-1-indanon)-2-yl)methylpiperidine; 1-benzyl-4-((5,6-dimethoxy-1-indanon)-2-yl)propylpiperidine; 1-benzyl-4-((5-isopropoxy-6-methoxy-1-indanon)-2-yl)methylpiperidine; 1-benzyl-4-((5,6-dimethoxy-1-oxoindanon)-2-yl)propenylpiperidine; pharmaceutically acceptable salts of one or more of the foregoing; stereoisomers of one or more of the foregoing; or pharmaceutically acceptable salts of stereoisomers of one or more of the foregoing.

In other embodiments, the compound of formula (VI) used in the methods and compositions described herein is preferably 1-benzyl-4-((5,6-dimethoxy-1-indanon)-2-yl)methylpiperidine; which is represented by formula (B):

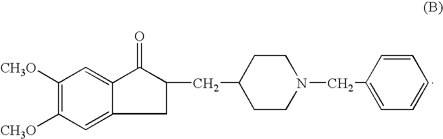

The compound of Formula (B), known as donepezil, may be in the form of a polymorph or a polymorphic crystal. For example, donepezil may be in the form of polymorph (II), (III), (IV), or (V); preferably polymorph (III). Donepezil may be in the form of polymorphic crystals (A), (B), or (C). Polymorphs, polymorphic crystals, and methods for making polymorphs and polymorphic crystals are described in U.S. Pat. Nos. 5,985,864, 6,140,321 and 6,245,911, the disclosures of which are incorporated by reference herein in their entirety.

In still other embodiments, the compound of formula (III) is 1-benzyl-4-((5,6-dimethoxy-1-indanon)-2-yl)methylpiperidine hydrochloride, which is also known as donepezil hydrochloride, and which is represented by formula (B 1):

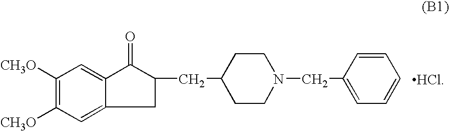

The compounds of the invention can have an asymmetric carbon atom(s), depending upon the substituents, and can have stereoisomers, which are within the scope of the invention. For example, donepezil or pharmaceutically acceptable salts thereof can be in the forms described in Japanese Patent Application Nos. 4-187674 and 4-21670, the disclosures of which are incorporated by reference herein in their entirety.

Japanese Patent Application No. 4-187674 describes a compound of formula (B2): 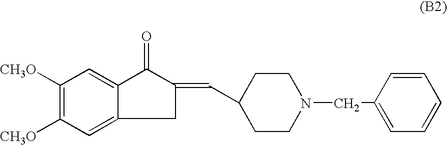
which can be in the form of a pharmaceutically acceptable salt, such as a hydrochloride salt.

Japanese Patent Application No. 4-21670 describes compounds of formula (B3): 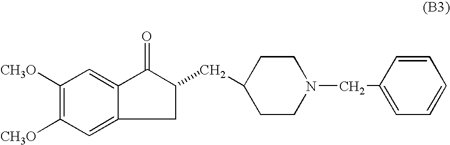
which can be in the form of a pharmaceutically acceptable salt, such as a hydrochloride salt; and compounds of formula (B4): 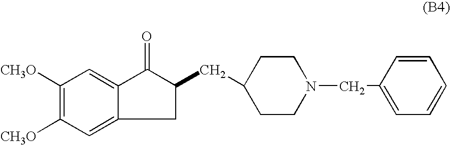
which can be in the form of a pharmaceutically acceptable salt, such as a hydrochloride salt; and compounds of formula (B5):

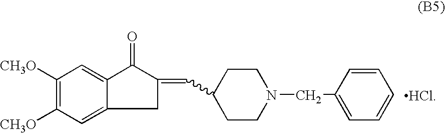

Throughout the specification, the terms “donepezil” and “1-benzyl-4-((5,6-dimethoxy-1-indanon)-2-yl)methylpiperidine” are intended to include one or more of the following (e.g., combinations of two or more thereof): pharmaceutically acceptable salts; stereoisomers; polymorphs; and polymorphic crystals.

The cholinesterase inhibitors of the invention are commercially available or can be prepared by processes known in the art, such as those described, for example, in U.S. Pat. Nos. 4,895,841, 5,985,864, 6,140,321 and 6,245,911; WO 98/39000, and Japanese Patent Application Nos. 4-187674 and 4-21670, the disclosures of which are incorporated by reference herein in their entirety.

The anti-migraine agents used in the methods and compositions of the invention may be any known in the art. Anti-migraine agents include, for example, serotonin antagonists, non-steroidal antiinflammatory drugs (NSAIDs) (e.g., COX-1 inhibitors and/or COX-2 inhibitors), calcium channel blockers, beta-andrenergic blockers, anticonvulsants, and antidepressants (e.g., tricylcic antidepressants, monoamine oxidase inhibitors, and selective serotonin reuptake inhibitors). Other exemplary anti-migraine agents include PNU-142633, vigabatrin, topiramate, montelukast (preferably the sodium salt thereof), gabapentin, piroxicam (preferably piroxicam betadex), valproate (preferably the semisodium salt thereof), diclofenac (preferably the potassium salt), tiagabine, botulinum, nebivolol, lisinopril, nimodipine, tizanidine, zolmitriptan, sumatriptan (preferably the succinate salt thereof), rizatriptan (preferably the benzoate salt thereof), pizotifen, oxetorone, naratriptan, lomerizine (preferably the hydrochloride salt thereof), gepefrine, flunarizine, almotriptan, alpiropride, tolfenamic acid, migpriv, timolol (preferably the maleate salt thereof), buclizine (preferably the hydrochloride salt thereof), baclofen, methysergide (preferably the maleate salt thereof), flunarizine (preferably the hydrochloride salt thereof), cyproheptadine (preferably the hydrochloride salt thereof), ergotamine (preferably the tartrate salt thereof), lidocaine (preferably the hydrochloride salt thereof), indoramin (preferably the hydrochloride salt thereof), butorphanol, KT 2962, BMS 181885, ADDS-ergotamine, NPS-1776, GW-468816, triptan, Pharmaprojects No. 6313, MT-500, donitriptan (preferably the mesylate salt thereof), ALX-0646, civamide, propanolol, zucapsaicin, CNS 5161, vofopitant, lanepitant, dapitant, ganaxolone, LY-53857, sergolexole (preferably the maleate salt thereof), sumatriptan, MT-400, fluoxetine, (S)-fluoxetine, dihydroergotamine (preferably the mesylate salt thereof), tonabersat, IS-159, BIBN-4096, metoclopramide, naproxen, MT-100 (i.e., a combination of metoclopramide and naproxen), dotarizine, frovatriptan, eletriptan, aspirin, ibuprofen, acetaminophen, amitryptiline, doxepin, ergot preparations, caffeine, cafergot (e.g., a combination of caffeine and ergotamine), codeine, meperidine, promethazine, atropine, phenobarbital, nifedipine, verapamil, chlorpromazine, lithium, prednisone, propranolol, phenelzine, mefenamic acid, flufenamic acid, LY334370, indomethacin, dichloralphenazone, isometheptene, butalbital, ketorolac, clonazepam, atenolol, metoprolol, nadolol, imipramine, nortripyline, diltiazem, valproic acid, divalproex, or cyproheptadine. Anti-migraine agents are commercially available or can be prepared by methods well known in the literature.

In other embodiments, the invention provides pharmaceutical compositions comprising a therapeutically effective amount of: (i) at least one 1,2-dihydropyridine compound, (ii) at least one cholinesterase inhibitor, and (iii) at least one pharmaceutically acceptable excipient. The invention also provides combinations comprising a therapeutically effective amount of: (i) at least one 1,2-dihydropyridine compound and (ii) at least one cholinesterase inhibitor; wherein the compounds may be administered separately (e.g., simultaneously, sequentially) to a patient to treat the diseases or disorders described. The invention provides commercial packages (e.g., kits) comprising a therapeutically effective amount of: (i) at least one 1,2-dihydropyridine compound, (ii) at least one cholinesterase inhibitor; and (iii) instructions for the simultaneous, separate or sequential use of (i) and (ii) in the treatment of the diseases and disorders described herein. The 1,2-dihydropyridine compound can be any described herein. For example, the 1,2-dihydropyridine compound can be a compound of Formula (I), a compound of Formula (II), a compound of Formula (III), or Compound A. The cholinesterase inhibitor can be any described herein. For example, the cholinesterase inhibitor can be a compound of Formula (IV), a compound of Formula (V), a compound of Formula (VI), a compound of Formula (B), a compound of Formula (B1), a compound of Formula (B2), a compound of Formula (B3), a compound of Formula (B4), or a compound of Formula (B5). In other embodiments, the cholinesterase inhibitor can be tacrine, physostigmine, pyridostigmine, neostigmine, rivastigmine, galantamine, citicoline, velnacrine, huperzine (e.g., huperzine A), metrifonate, heptastigmine, edrophonium, phenserine, tolserine, phenethylnorcymserine, quilostigmine, ganstigmine, epastigmine, upreazine, 3-[1-(phenylmethyl)-4-piperidinyl]-1-(2,3 ,4,5-tetrahydro-1H-1-benzazepin-8-yl)-1-propanone, or (2-[2-(1-benzylpiperidin-4-yl)ethyl]-2,3-dihydro-9-methoxy-1H-pyrrolo [3,4-b]quinolin-1-one. In one embodiment, the invention provides pharmaceutical compositions comprising a therapeutically effective amount of: (i) donepezil; (ii) 3-(2-cyanophenyl)-5-(2-pyridyl)-1-phenyl-1,2-dihydropyridin-2-one; and (iii) at least one pharmaceutically acceptable excipient. The pharmaceutical compositions can optionally further comprise at least one anti-migraine agent.

The invention provides pharmaceutical compositions comprising a therapeutically effective amount of: (i) at least on 1,2-dihydropyridine compound, (ii) at least one anti-migraine agent, and (iii) at least one pharmaceutically acceptable excipient. The 1,2-dihydropyridine compound can be any described herein. For example, the 1,2-dihydropyridine compound can be a compound of Formula (I), a compound of Formula (II), a compound of Formula (III), or a compound of Formula (A). The anti-migraine agent can be any described herein. For example, the anti-migraine agent can be aspirin, ibuprofen, acetaminophen, diclofenac, fenoprofen, ketaprofen, ketorolac, flurbiprofen, meclofenamate, naproxen, ergotamine, sumatriptan, zolmitriptan, rizatriptan, naratriptin, almotriptin, frovaltriptan, eletriptan, amitriptyline, desipramine, doxepin, imipramine, nortripytyline, fluoxetine, paroxetine, sertraline, venlafazine, trazodone, bupropion, atenolol, metoprolol, nadolol, propranolol, timolol, diltiazem, nicardipine, nifedipine, nimodipine, verapamil, divalproex, gabapentin, valproic acid, or topiramate. In one embodiment, the anti-migraine agent is aspirin, ibuprofen, acetaminophen, or naproxen. In another embodiment, the anti-migraine agent is ergotamine, dihydroergotamine, sumatriptan, zolmitriptan, rizatriptan, naratriptan or almotriptan. The pharmaceutical compositions can optionally further comprise at least one cholinesterase inhibitor.

The invention provides methods for the treatment and/or prophylaxis of headaches in a patient in need thereof by administering a therapeutically effective amount of: (a) at least one 1,2-dihydropyridine compound, and optionally (b); wherein (b) is a therapeutically effective amount of (i) at least one cholinesterase inhibitor; (ii) at least one anti-migraine agent; or (iii) at least one cholinesterase inhibitor and at least one anti-migraine agent. The methods for the treatment of headaches includes (i) methods for reducing the frequency of headaches, (ii) methods for reducing the severity of headaches, (iii) methods for reducing the duration of headaches, (iv) methods for reducing the frequency and severity of headaches, (v) methods for reducing the frequency and duration of headaches, (vi) methods for reducing the severity and duration of headaches, and (vii) methods for reducing the frequency, severity and duration of headaches. The methods for the treatment of headaches includes methods of treating of one or more symptoms caused by headaches. The headaches may be primary headaches or secondary headaches. The 1,2-dihydropyridine compound and the cholinesterase inhibitor and/or anti-migraine agent can be administered separately to the patient or may be administered in the form of a pharmaceutical composition.

The invention provides methods for the treatment and/or prophylaxis of primary headaches or secondary headaches in a patient in need thereof comprising administering a therapeutically effective amount of: (a) at least one 1,2-dihydropyridine compound, and optionally (b); wherein (b) is a therapeutically effective amount of: (i) at least one cholinesterase inhibitor; (ii) at least one anti-migraine agent; or (iii) at least one cholinesterase inhibitor and at least one anti-migraine agent. The methods for the treatment of primary headaches or secondary headaches includes (i) methods for reducing the frequency of primary headaches or secondary headaches, (ii) methods for reducing the severity of primary headaches or secondary headaches, (iii) methods for reducing the duration of primary headaches or secondary headaches, (iv) methods for reducing the frequency and severity of primary headaches or secondary headaches, (v) methods for reducing the frequency and duration of primary headaches or secondary headaches, (vi) methods for reducing the severity and duration of primary headaches or secondary headaches, and (vii) methods for reducing the frequency, severity and duration of primary headaches or secondary headaches. The methods for the treatment of primary headaches or secondary headaches includes methods of treating of one or more symptoms caused by primary headaches or secondary headaches. The 1,2-dihydropyridine compound and, optionally, the cholinesterase inhibitor and/or anti-migraine agent can be administered separately to the patient or may be administered in the form of a pharmaceutical composition.

The invention provides methods for the treatment and/or prophylaxis of migraines in a patient in need thereof by administering a therapeutically effective amount of: (a) at least one 1,2-dihydropyridine compound, and optionally (b); wherein (b) is a therapeutically effective amount of: (i) at least one cholinesterase inhibitor; (ii) at least one anti-migraine agent; or (iii) at least one cholinesterase inhibitor and at least one anti-migraine agent. The methods for the treatment of migraines include (i) methods for reducing the frequency of migraines, (ii) methods for reducing the severity of migraines, (iii) methods for reducing the duration of migraines, (iv) methods for reducing the frequency and severity of migraines, (v) methods for reducing the frequency and duration of migraines, (vi) methods for reducing the severity and duration of migraines, and (vii) methods for reducing the frequency, severity and duration of migraines. The methods for the treatment of migraines includes methods of treating of one or more symptoms caused by migraines. The 1,2-dihydropyridine compound and the cholinesterase inhibitor and/or anti-migraine agent can be administered separately to the patient or may be administered in the form of a pharmaceutical composition.

The invention provides methods for the treatment and/or prophylaxis of classic migraines in a patient by administering a therapeutically effective amount of: (a) at least one 1,2-dihydropyridine compound, and optionally (b); wherein (b) is a therapeutically effective amount of: (i) at least one cholinesterase inhibitor; (ii) at least one anti-migraine agent; or (iii) at least one cholinesterase inhibitor and at least one anti-migraine agent. The methods for treating classic migraines include (i) methods for reducing the frequency of classic migraines, (ii) methods for reducing the severity of classic migraines, (iii) methods for reducing the duration of classic migraines, (iv) methods for reducing the frequency and severity of classic migraines, (v) methods for reducing the frequency and duration of classic migraines, (vi) methods for reducing the severity and duration of classic migraines, and (vii) methods for reducing the frequency, severity and duration of classic migraines. The methods for the treatment of classic migraines includes methods of treating of one or more symptoms caused by classic migraines. “Classic migraines” generally begin with neurologic symptoms such as visual scintillations, dazzling zigzag lines, photophobia and spreading scotomas, or dizziness and tinnitus. Classic migraines can have premonitory symptoms such as feelings of elation, excessive energy, thirst, cravings for sweets, and/or drowsiness. At other times, classic migraines can have premonitory symptoms such as a slowing of mentation, a feeling of impending doom, and/or depression. At other times, there can be no premonitory symptoms.

The invention provides methods for the treatment and/or prophylaxis of common migraines in a patient by administering a therapeutically effective amount of: (a) at least one 1,2-dihydropyridine compound, and optionally (b); wherein (b) is a therapeutically effective amount of: (i) at least one cholinesterase inhibitor; (ii) at least one anti-migraine agent; or (iii) at least one cholinesterase inhibitor and at least one anti-migraine agent. The methods for treating common migraines include (i) methods for reducing the frequency of common migraines, (ii) methods for reducing the severity of common migraines, (iii) methods for reducing the duration of common migraines, (iv) methods for reducing the frequency and severity of common migraines, (v) methods for reducing the frequency and duration of common migraines, (vi) methods for reducing the severity and duration of common migraines, and (vii) methods for reducing the frequency, severity and duration of common migraines. The methods for the treatment of common migraines includes methods of treating of one or more symptoms caused by common migraines. “Common migraines” generally have an unheralded onset of headache that can be accompanied by nausea and/or vomiting. Unlike the classic migraine, the common migraine generally does not have neurologic symptoms that occur prior to the onset of the headache.

The invention provides methods for the treatment and/or prophylaxis of complicated migraines in a patient by administering a therapeutically effective amount of: (a) at least one 1,2-dihydropyridine compound, and optionally (b); wherein (b) is a therapeutically effective amount of: (i) at least one cholinesterase inhibitor; (ii) at least one anti-migraine agent; or (iii) at least one cholinesterase inhibitor and at least one anti-migraine agent. The methods for treating complicated migraines include (i) methods for reducing the frequency of complicated migraines, (ii) methods for reducing the severity of complicated migraines, (iii) methods for reducing the duration of complicated migraines, (iv) methods for reducing the frequency and severity of complicated migraines, (v) methods for reducing the frequency and duration of complicated migraines, (vi) methods for reducing the severity and duration of complicated migraines, and (vii) methods for reducing the frequency, severity and duration of complicated migraines. The methods for the treatment of complicated migraines includes methods of treating of one or more symptoms caused by complicated migraines. “Complicated migraines” refers to migraines accompanied by neurologic symptoms (e.g., such as those described for classic migraines) that can either precede or accompany the headache. In complicated migraines, numbness and tingling of the lips, face, hand, arm, and/or leg on side of the body can occur, sometimes in combination with aphasic disorder. The arm and/or leg can become weak or paralyzed on one side, mimicking a stroke. The numbness or weakness can spread from one part of the body to another slowly over a period of minutes. “Complicated migraines” include basilar migraines. In basilar migraines, the visual disorder and paresthesias are bilateral and can be accompanied by confusion, stupor, coma, aggressive outbursts, vertigo, diplopia, and/or dysarthria. Basilar migraines occur in 30% of children with migraines.

The invention provides methods for the treatment and/or prophylaxis of menstrual migraines or premenstrual migraines in a patient by administering a therapeutically effective amount of: (a) at least one 1,2-dihydropyridine compound, and optionally (b); wherein (b) is a therapeutically effective amount of: (i) at least one cholinesterase inhibitor; (ii) at least one anti-migraine agent; or (iii) at least one cholinesterase inhibitor and at least one anti-migraine agent. The methods for treating menstrual migraines or premenstrual migraines include (i) methods for reducing the frequency of menstrual migraines or premenstrual migraines, (ii) methods for reducing the severity of menstrual migraines or premenstrual migraines, (iii) methods for reducing the duration of menstrual migraines or premenstrual migraines, (iv) methods for reducing the frequency and severity of menstrual migraines or premenstrual migraines, (v) methods for reducing the frequency and duration of menstrual migraines or premenstrual migraines, (vi) methods for reducing the severity and duration of menstrual migraines or premenstrual migraines, and (vii) methods for reducing the frequency, severity and duration of menstrual migraines or premenstrual migraines. The methods for the treatment of menstrual migraines or premenstrual migraines includes methods of treating of one or more symptoms caused by menstrual migraines or premenstrual migraines. “Menstrual migraines” refer to migraine headaches that can generally occur from about 2 days prior to a woman's menstrual cycle until about 3 days after a woman's menstrual cycle. In another embodiment, menstrual migraines refer to migraine headaches that can generally occur from about 2 days prior to a woman's menstrual cycle and that generally end on the last day of the woman's menstrual cycle. Menstrual migraines can occur or re-occur at any time during the menstrual cycle. “Premenstrual migraines” are migraine headaches that can generally occur from about 7 days prior to a woman's menstrual cycle to about 3 days prior to a woman's menstrual cycle. Premenstrual migraines can occur or re-occur at any time during the premenstrual cycle.

The invention provides methods for the treatment and/or prophylaxis of ophthalmic migraines or ophthalmoplegic migraines in a patient by administering a therapeutically effective amount of: (a) at least one 1,2-dihydropyridine compound, and optionally (b); wherein (b) is a therapeutically effective amount of: (i) at least one cholinesterase inhibitor; (ii) at least one anti-migraine agent; or (iii) at least one cholinesterase inhibitor and at least one anti-migraine agent. The methods for treating ophthalmic migraines or ophthalmoplegic migraines include (i) methods for reducing the frequency of ophthalmic migraines or ophthalmoplegic migraines, (ii) methods for reducing the severity of ophthalmic migraines or ophthalmoplegic migraines, (iii) methods for reducing the duration of ophthalmic migraines or ophthalmoplegic migraines, (iv) methods for reducing the frequency and severity of ophthalmic migraines or ophthalmoplegic migraines, (v) methods for reducing the frequency and duration of ophthalmic migraines or ophthalmoplegic migraines, (vi) methods for reducing the severity and duration of ophthalmic migraines or ophthalmoplegic migraines, and (vii) methods for reducing the frequency, severity and duration of ophthalmic migraines or ophthalmoplegic migraines. The methods for the treatment of ophthalmic migraines or ophthalmoplegic migraines includes methods of treating of one or more symptoms caused by ophthalmic migraines or ophthalmoplegic migraines. “Ophthalmic migraines” are migraine headaches that are generally accompanied by a marked disturbance of vision. “Ophthalmoplegic migraines” are migraine headaches associated with paralysis of the eye muscles.

The invention provides methods for the treatment and/or prophylaxis of fulgurating migraines in a patient by administering a therapeutically effective amount of: (a) at least one 1,2-dihydropyridine compound, and optionally (b); wherein (b) is a therapeutically effective amount of: (i) at least one cholinesterase inhibitor; (ii) at least one anti-migraine agent; or (iii) at least one cholinesterase inhibitor and at least one anti-migraine agent. The methods for treating fulgurating migraines include (i) methods for reducing the frequency of fulgurating migraines, (ii) methods for reducing the severity of fulgurating migraines, (iii) methods for reducing the duration of fulgurating migraines, (iv) methods for reducing the frequency and severity of fulgurating migraines, (v) methods for reducing the frequency and duration of fulgurating migraines, (vi) methods for reducing the severity and duration of fulgurating migraines, and (vii) methods for reducing the frequency, severity and duration of fulgurating migraines. The methods for the treatment of fulgurating migraines includes methods of treating of one or more symptoms caused by fulgurating migraines. “Fulgurating migraines” are migraine headaches characterized by an abrupt beginning and severity.

The invention provides methods for the treatment and/or prophylaxis of Harris' migraines in a patient by administering a therapeutically effective amount of: (a) at least one 1,2-dihydropyridine compound, and optionally (b); wherein (b) is a therapeutically effective amount of: (i) at least one cholinesterase inhibitor; (ii) at least one anti-migraine agent; or (iii) at least one cholinesterase inhibitor and at least one anti-migraine agent. The methods for treating Harris' migraines include (i) methods for reducing the frequency of Harris' migraines, (ii) methods for reducing the severity of Harris' migraines, (iii) methods for reducing the duration of Harris' migraines, (iv) methods for reducing the frequency and severity of Harris' migraines, (v) methods for reducing the frequency and duration of Harris' migraines, (vi) methods for reducing the severity and duration of Harris' migraines, and (vii) methods for reducing the frequency, severity and duration of Harris' migraines. The methods for the treatment of Harris' migraines includes methods of treating of one or more symptoms caused by Harris' migraines. “Harris' migraine” is also known as periodic migrainous neuralgia.

The invention provides methods for the treatment and/or prophylaxis of hemiplegic migraines in a patient by administering a therapeutically effective amount of: (a) at least one 1,2-dihydropyridine compound, and optionally (b); wherein (b) is a therapeutically effective amount of: (i) at least one cholinesterase inhibitor; (ii) at least one anti-migraine agent; or (iii) at least one cholinesterase inhibitor and at least one anti-migraine agent. The methods for treating hemiplegic migraines include (i) methods for reducing the frequency of hemiplegic migraines, (ii) methods for reducing the severity of hemiplegic migraines, (iii) methods for reducing the duration of hemiplegic migraines, (iv) methods for reducing the frequency and severity of hemiplegic migraines, (v) methods for reducing the frequency and duration of hemiplegic migraines, (vi) methods for reducing the severity and duration of hemiplegic migraines, and (vii) methods for reducing the frequency, severity and duration of hemiplegic migraines. The methods for the treatment of hemiplegic migraines includes methods of treating of one or more symptoms caused by hemiplegic migraines. “Hemiplegic migraines” are a form of migraine headache associated with transient hemiplegia.

The invention provides methods for the treatment and/or prophylaxis of abdominal migraines in a patient by administering a therapeutically effective amount of: (a) at least one 1,2-dihydropyridine compound, and optionally (b); wherein (b) is a therapeutically effective amount of: (i) at least one cholinesterase inhibitor; (ii) at least one anti-migraine agent; or (iii) at least one cholinesterase inhibitor and at least one anti-migraine agent. The methods for treating abdominal migraines include (i) methods for reducing the frequency of abdominal migraines, (ii) methods for reducing the severity of abdominal migraines, (iii) methods for reducing the duration of abdominal migraines, (iv) methods for reducing the frequency and severity of abdominal migraines, (v) methods for reducing the frequency and duration of abdominal migraines, (vi) methods for reducing the severity and duration of abdominal migraines, and (vii) methods for reducing the frequency, severity and duration of abdominal migraines. The methods for the treatment of abdominal migraines includes methods of treating of one or more symptoms caused by abdominal migraines. “Abdominal migraines” are characterized by paroxysmal abdominal pain without apparent cause.

The invention provides methods for the treatment and/or prophylaxis of cluster headaches in a patient by administering a therapeutically effective amount of: (a) at least one 1,2-dihydropyridine compound, and optionally (b); wherein (b) is a therapeutically effective amount of: (i) at least one cholinesterase inhibitor; (ii) at least one anti-migraine agent; or (iii) at least one cholinesterase inhibitor and at least one anti-migraine agent. The methods for treating cluster headaches include (i) methods for reducing the frequency of cluster headaches, (ii) methods for reducing the severity of cluster headaches, (iii) methods for reducing the duration of cluster headaches, (iv) methods for reducing the frequency and severity of cluster headaches, (v) methods for reducing the frequency and duration of cluster headaches, (vi) methods for reducing the severity and duration of cluster headaches, and (vii) methods for reducing the frequency, severity and duration of cluster headaches. The methods for the treatment of cluster headaches includes methods of treating of one or more symptoms caused by cluster headaches. “Cluster headaches” are also called paroxysmal nocturnal cephalalgia, migrainous neuralgia, histamine headache, and Horton's syndrome. Cluster headaches are characterized by constant, unilateral orbital pain, with onset usually within two or three hours after falling asleep. The pain can be intense and steady with lacrimation, blocked nostril, then rhinorrhea, and sometimes miosis, ptosis, flush, and edema of cheek.

In other embodiments, the invention provides methods for treating and/or preventing panic attacks and/or panic disorders by administering a therapeutically effective amount of at least one 1,2-dihydropyridine compound, and, optionally at least on anxiolytic agent. The anxiolytic agent can be any in the art. Exemplary anxiolytic agent include benzodiazepines, azaspirodecanediones, or piperazine derivatives. Exemplary benzodiazepines include diazepam, alprazolam, chlordiazepoxide, clonazepam, clorazepate, halazepam, lorpam, oxazepam, derivatives thereof, and pharmaceutically acceptable salts thereof. Exemplary azaspirodecanediones include buspirone, derivatives thereof, and pharmaceutically acceptable salts thereof. Exemplary piperazine derivatives include hydroxyzine pamoate and hydroxyzine hydrochloride, derivatives thereof, and pharmaceutically acceptable salts thereof. The 1,2-dihydropyridine compound and the anxiolytic agents can be administered separately or can be administered in the form of a composition.

The invention provides methods for treating and/or preventing acute stress disorders and/or generalized anxiety disorders by administering a therapeutically effective amount of at least one 1,2-dihydropyridine compound, and, optionally at least one anxiolytic agent. The anxiolytic agent can be any in the art. Exemplary anxiolytic agent include benzodiazepines, azaspirodecanediones, or piperazine derivatives. Exemplary benzodiazepines include diazepam, alprazolam, chlordiazepoxide, clonazepam, clorazepate, halazepam, lorpam, oxazepam, derivatives thereof, and pharmaceutically acceptable salts thereof. Exemplary azaspirodecanediones include buspirone, derivatives thereof, and pharmaceutically acceptable salts thereof. Exemplary piperazine derivatives include hydroxyzine pamoate and hydroxyzine hydrochloride, derivatives thereof, and pharmaceutically acceptable salts thereof. The 1,2-dihydropyridine compound and the anxiolytic agents can be administered separately or can be administered in the form of a composition.

The invention provides methods for sedating a patient by administering a therapeutically effective amount of at least one 1,2-dihydropyridine compound. In one embodiment, the invention provides methods for sedating a patient prior to surgery by administering a therapeutically effective amount of at least one 1,2-dihydropyridine compound. In another embodiment, the invention provides methods for acute sedation for surgical anesthesia in a patient in need thereof patient by administering a therapeutically effective amount of at least one 1,2-dihydropyridine compound.

The 1,2-dihydropyridine compounds and other compounds of the invention (e.g., cholinesterase inhibitors, anti-migraine agents, anxiolytic agents) can be administered orally, topically, parenterally, by inhalation (nasal or oral), or rectally in dosage unit formulations containing conventional nontoxic pharmaceutically acceptable carriers, adjuvants, and vehicles as desired. The term parenteral includes subcutaneous, intravenous, intramuscular, intrathecal, intrasternal injection, or infusion techniques.

The daily dose of the 1,2-dihydropyridine compounds of the invention (e.g., 3-(2-cyanophenyl)-5-(2-pyridyl)-1-phenyl-1,2-dihydropyridin-2-one) is usually about 30 μg to 10 grams, preferably, 100 μg to 5 grams or, more preferably, 100 μg to 100 mg in the case of oral administration. For administration by injection, the daily dose is usually about 30 μg to 1 gram, preferably 100 μg to 500 mg or, more preferably, 100 μg to 30 mg. The compounds are administered once daily or in several portions a day. When used in the context of a dosage amount, the numerical weight refers to the weight of the 1,2-dihydropyridine, exclusive of any salt, counterion, hydrate, and the like. Therefore to obtain the equivalent of 500 milligrams of 3-(2-cyanophenyl)-5-(2-pyridyl)-1-phenyl-1,2-dihydropyridin-2-one, it would be necessary to use more than 500 milligrams of a pharmaceutically acceptable salt and/or hydrate of the compound, due to the additional weight of the pharmaceutically acceptable salt and/or hydrate.

The daily dose of the cholinesterase inhibitors of the invention (e.g., donepezil) are usually about 0.1 milligram to 100 milligrams, preferably 1 milligram to 50 milligrams, more preferably 5 milligrams to 25 milligrams. In other embodiment, the daily dose is from 10 milligrams to 20 milligrams; or from 5 milligrams to 10 milligrams. The compounds are administered once daily or in several portions a day. When used in the context of a dosage amount, the numerical weight refers to the weight of the cholinesterase inhibitors of the invention, exclusive of any salt, counterion, and the like. Therefore to obtain the equivalent of 10 milligrams of donepezil, it would be necessary to use more than 10 milligrams of donepezil hydrochloride, due to the additional weight of the hydrochloride.

The other compounds described herein (e.g., anti-migraine agents, anxiolytic agents) may be administered in doses well known in the art by reference, for example, to The Physician's Desk Reference, to patents describing doses for the compounds, and to journal articles describing doses for the compounds.

In one embodiment, the mode of administration is by injection, such as subcutaneous injection, intramuscular injection, intravenous injection, or intra-arterial injection. Injectable preparations, for example, sterile injectable aqueous or oleaginous suspensions can be formulated according to the art using suitable dispersing or wetting agents, suspending agents (e.g., methylcellulose, Polysorbate 80, hydroxyethylcellulose, acacia, powdered tragacanth, sodium carboxymethylcellulose, polyoxytehylene sorbitan monolaurate and the like), pH modifiers, buffers, solubilizing agents (e.g., polyoxyethylene hydrogenated castor oil, Polysorbate 80, nicotinamide, polyoxyethylene sorbitan monolaurate, Macrogol, an ethyl ester of castor oil fatty acid, and the like) and preservatives. The sterile injectable preparation can also be a sterile injectable solution or suspension in a nontoxic parenterally acceptable diluent or solvent, for example, as a solution in 1,3-butanediol. Among the acceptable vehicles and solvents that can be used are water, Ringer's solution, and isotonic sodium chloride solution. In addition, sterile, fixed oils are conventionally used as a solvent or suspending medium. For this purpose any bland fixed oil can be used including synthetic mono- or diglycerides, in addition, fatty acids, such as oleic acid, can be used in the preparation of injectables. The preparations can be lyophilized by methods known in the art.

Solid dosage forms for oral administration can include chewing gum, capsules, tablets, sublingual tablets, powders, granules, and gels. In such solid dosage forms, the active compound can be admixed with one or more inert diluents such as lactose or starch. As is normal practice, such dosage forms can also comprise other substances including lubricating agents such as magnesium stearate. In the case of capsules, tablets, and pills, the dosage forms can also comprise buffering agents. The tablets can be prepared with enteric or film coatings, preferably film coatings.

To make tablets, the compounds can be admixed with pharmaceutically acceptable carriers known in the art such as, for example, vehicles (e.g., lactose, white sugar, mannitol, glucose, starches, calcium carbonate, crystalline cellulose, silicic acid, and the like), binders (e.g., water, ethanol, myranol, glucose solution, starch solution, gelatin solution, polyvinylpyrrolidone, and the like), disintegrators (e.g., dry starch, sodium, alginate, sodium hydrogen carbonate, calcium carbonate, polyoxyethylene sorbitan fatty acid esters, sodium laurylsulfate, stearic monoglyceride, starches, lactose, and the like), absorption promoters (e.g., quaternary ammonium base, sodium laurylsulfate, and the like), wetting agents (e.g. glycerin, starches, and the like), lubricants (e.g., stearates, polyethylene glycol, and the like), and flavoring agents (e.g., sweeteners). The tablets can be in the form of a conventional tablet, a molded tablet, a wafer and the like.

Sublingual administration refers to the administration in the mouth (e.g., under the tongue, between the cheek and gum, between the tongue and roof of the mouth). The highly vascular mucosal lining in the mouth is a convenient location for the compounds to be administered into the body.

In other embodiments, the solid dosage form can be packaged as granules or a powder in a pharmaceutically acceptable carrier, where the granules or powder are removed from the packaging and sprinkled on food or mixed with a liquid, such as water or juice, or where the granules are inserted into capsules. In this embodiment, the compounds described herein can be mixed with flavoring or sweetening agents. The packaging material can be plastic, coated paper, or any material that prevents water or moisture from reaching the granules and/or powder.

Liquid dosage forms for oral administration can include pharmaceutically acceptable emulsions, solutions, sublingual solutions, suspensions, and syrups containing inert diluents commonly used in the art, such as water. Such compositions can also comprise adjuvants, such as wetting agents, emulsifying and suspending agents, and sweetening, flavoring, and perfuming agents. To make sublingual solutions, the compounds can be admixed with various carriers, excipients, pH adjusters, and the like (e.g., water, sugar, lactic acid, acetic acid, fructose, glucose, saccharin, polyethylene glycol, propylene glycol, alcohol, bentonite, tragacanth, gelatin, alginates, aspartame, sorbitol, methylparaben, propylparaben, sodium benzoate, artificial flavoring and coloring agents).

For administration by inhalation, the compounds can be delivered from an insufflator, a nebulizer or a pressured pack or other convenient mode of delivering an aerosol spray. Pressurized packs can include a suitable propellant. Alternatively, for administration by inhalation, the compounds can be administered in the form of a dry powder composition or in the form of a liquid spray.

Suppositories for rectal administration can be prepared by mixing the active compounds with suitable nonirritating excipients such as cocoa butter and polyethylene glycols that are solid at room temperature and liquid at body temperature. Alternatively, an enema can be prepared by for rectal administration of the compounds described herein.

For topical administration to the epidermis, the compounds can be formulated as ointments, creams or lotions, or as the active ingredient of a transdermal patch. The compounds can also be administered via iontophoresis or osmotic pump. Ointments, creams and lotions can be formulated with an aqueous or oily base with the addition of suitable thickening and/or gelling agents. Alternatively, ointments, creams and lotions can be formulated with an aqueous or oily base and can also contain one or more emulsifying agents, stabilizing agents, dispersing agents, suspending agents, thickening agents, and/or coloring agents. As creams or lotions, the compounds can be mixed to form a smooth, homogeneous cream or lotion with, for example, one or more of a preservative (e.g., benzyl alcohol 1% or 2% (wt/wt)), emulsifying wax, glycerin, isopropyl palmitate, lactic acid, purified water, sorbitol solution. Such topically administrable compositions can contain polyethylene glycol 400. To form ointments, the compounds can be mixed with one or more of a preservative (e.g., benzyl alcohol 2% (wt/wt)), petrolatum, emulsifying wax, and Tenox (II) (e.g., butylated hydroxyanisole, propyl gallate, citric acid, propylene glycol). Woven pads or rolls of bandaging material, e.g., gauze, can be impregnated with the transdermally administrable compositions for topical application.

The compounds can also be topically applied using a transdermal system, such as one of an acrylic-based polymer adhesive with a resinous crosslinking agent impregnated with the compounds described herein and laminated to an impermeable backing. For example, the compounds can be administered in the form of a transdermal patch, such as a sustained-release transdermal patch. Transdermal patches can include any conventional form such as, for example, an adhesive matrix, a polymeric matrix, a reservoir patch, a matrix- or monolithic-type laminated structure, and are generally comprised of one or more backing layers, adhesives, penetration enhancers, and/or rate-controlling membranes. Transdermal patches generally have a release liner which is removed to expose the adhesive/active ingredient(s) prior to application. Transdermal patches are described in, for example, U.S. Pat. Nos. 5,262,165, 5,948,433, 6,010,715 and 6,071,531, the disclosures of which are incorporated by reference herein in their entirety.

The invention provides for the compounds to be administered nasally to a patient to treat the diseases and disorders described herein. “Administered nasally” or “nasal administration” is intended to mean that at least one compound is combined with a suitable delivery system for absorption across the nasal mucosa of a patient. Generally, lower doses of the compound can be used for nasal administration when compared, for example, to the dose required for the oral administration.

The compounds can be administered, for example, as nasal sprays, nasal drops, nasal suspensions, nasal gels, nasal ointments, nasal creams or nasal powders. The compounds can also be administered using nasal tampons or nasal sponges. The compounds can be brought into a viscous basis via systems conventionally used, for example, natural gums, methylcellulose and derivatives, acrylic polymers (carbopol) and vinyl polymers (polyvinylpyrrolidone). In the compositions, many other excipients known in the art can be added such as water, preservatives, surfactants, solvents, adhesives, antioxidants, buffers, bio-adhesives, viscosity enhancing agents and agents to adjust the pH and the osmolarity.

The nasal delivery systems can take various forms including aqueous solutions, non-aqueous solutions and combinations thereof. Aqueous solutions include, for example, aqueous gels, aqueous suspensions, aqueous liposomal dispersions, aqueous emulsions, aqueous microemulsions and combinations thereof. Non-aqueous solutions include, for example, non-aqueous gels, non-aqueous suspensions, non-aqueous liposomal dispersions, non-aqueous emulsions, non-aqueous microemulsions and combinations thereof.

In other embodiments, the nasal delivery system can be a powder formulation. Powder formulations include, for example, powder mixtures, powder microspheres, coated powder microspheres, liposomal dispersions and combinations thereof. Preferably, the powder formulation is powder microspheres. The powder microspheres are preferably formed from various polysaccharides and celluloses selected from starch, methylcellulose, xanthan gum, carboxymethylcellulose, hydroxypropyl cellulose, carbomer, alginate polyvinyl alcohol, acacia, chitosans, and mixtures of two or more thereof.

In certain embodiments, the particle size of the droplets of the aqueous and/or non-aqueous solution or of the powders delivered to the nasal mucosa can be, for example, about 0.1 micron to about 100 microns; from about 1 micron to about 70 microns; from about 5 microns to about 50 microns; or from about 10 microns to about 20 microns. The particle sizes can be obtained using suitable containers or metering devices known in the art. Exemplary devices include mechanical pumps in which delivery is made by movement of a piston; compressed air mechanisms in which delivery is made by hand pumping air into the container; compressed gas (e.g., nitrogen) techniques in which delivery is made by the controlled release of a compressed gas in the sealed container; liquefied propellant techniques in which a low boiling liquid hydrocarbon (e.g., butane) is vaporized to exert a pressure and force the composition through the metered valve; and the like. Powders may be administered, for example, in such a manner that they are placed in a capsule that is then set in an inhalation or insufflation device. A needle is penetrated through the capsule to make pores at the top and the bottom of the capsule and air is sent to blow out the powder particles. Powder formulation can also be administered in a jet-spray of an inert gas or suspended in liquid organic fluids.

In one embodiment, the invention provides a nasally administrable pharmaceutical composition comprising at least one compound dispersed in a nasal delivery system that improves the solubility of the compound. The nasal delivery system that improves solubility can include one of the following or combinations thereof: (i) a glycol derivative (e.g., propylene glycol, polyethylene glycol, mixtures thereof); (ii) a sugar alcohol (e.g., mannitol, xylitol, mixtures thereof); (iii) glycerin; (iv) a glycol derivative (e.g., propylene glycol, polyethylene glycol or mixtures thereof) and glycerin; (v) ascorbic acid and water; (vi) sodium ascorbate and water; or (vii) sodium metabisulfite and water.

In another embodiment, the invention provides a nasally administrable pharmaceutical composition comprising at least one compound described herein and a nasal delivery system, where the nasal delivery system comprises at least one buffer to maintain the pH of the compound described herein, at least one pharmaceutically acceptable thickening agent and at least one humectant. The nasal delivery system can optionally further comprise surfactants, preservatives, antioxidants, bio-adhesives, pH adjusting agents, isotonicity agents, solubilizing agents, and/or other pharmaceutically acceptable excipients. The compound described herein can optionally be dispersed in a nasal delivery system that improves its solubility.

In another embodiment, the invention provides a nasally administrable pharmaceutical composition comprising at least one compound described herein and a nasal delivery system, where the nasal delivery system comprises at least one solubilizing agent, at least one pharmaceutically acceptable thickening agent and at least one humectant. The nasal delivery system can optionally further comprise buffers, pH adjusting agents, isotonicity agents, surfactants, preservatives, antioxidants, bio-adhesives, and/or other pharmaceutically acceptable excipients. The compound described herein can optionally be dispersed in a nasal delivery system that improves its solubility.

In another embodiment, the invention provides a nasally administrable pharmaceutical composition comprising at least one compound described herein and a nasal delivery system, where the nasal delivery system comprises at least one buffer to maintain the pH of the compound, at least one pharmaceutically acceptable thickening agent, at least one humectant, and at least one surfactant. The nasal delivery system can optionally further comprise pH adjusting agents, isotonicity agents, solubilizing agents, preservatives, antioxidants, bio-adhesives, and/or other pharmaceutically acceptable excipients. The compound described herein can optionally be dispersed in a nasal delivery system that improves its solubility.

In yet another embodiment, the invention provides a nasally administrable pharmaceutical composition comprising at least one compound described herein and a nasal delivery system, where the nasal delivery system comprises at least one pharmaceutically acceptable thickening agent, at least one humectant, at least one surfactant, and at least one solubilizing agent. The nasal delivery system can optionally further comprise buffers, pH adjusting agents, isotonicity agents, preservatives, antioxidants, bio-adhesives, and/or other pharmaceutically acceptable excipients. The compound can optionally be dispersed in a nasal delivery system that improves its solubility.

In yet another embodiment, the invention provides a nasally administrable pharmaceutical composition comprising at least one compound described herein and a nasal delivery system, where the nasal delivery system comprises at least one buffer to maintain the pH of the compound, at least one pharmaceutically acceptable thickening agent, at least one humectant, at least one surfactant, and at least one solubilizing agent. The nasal delivery system can optionally further comprise buffers, pH adjusting agents, isotonicity agents, preservatives, antioxidants, bio-adhesives, and/or other pharmaceutically acceptable excipients. The compound described herein can optionally be dispersed in a nasal delivery system that improves its solubility.

The buffer has a pH that is selected to optimize the absorption of the 1,2-dihydropyridine compound across the nasal mucosa. The particular pH of the buffer can vary depending upon the particular nasal delivery formulation as well as the specific compound selected. Buffers that are suitable for use in the invention include acetate (e.g., sodium acetate), citrate (e.g., sodium citrate dihydrate), phthalate, borate, prolamine, trolamine, carbonate, phosphate (e.g., monopotassium phosphate, disodium phosphate), and mixtures of two or more thereof.

The pH of the compositions should be maintained from about 3.0 to about 10.0. Compositions having a pH of less than about 3.0 or greater than about 10.0 can increase the risk of irritating the nasal mucosa of the patient. Further, it is preferable that the pH of the compositions be maintained from about 3.0 to about 9.0. With respect to the non-aqueous nasal formulations, suitable forms of buffering agents can be selected such that when the formulation is delivered into the nasal cavity of a mammal, selected pH ranges are achieved therein upon contact with, e.g., a nasal mucosa.

The solubilizing agent for use in the compositions of the invention can be any known in the art, such as carboxylic acids and salts thereof. Exemplary carboxylic acid salts include acetate, gluconate, ascorbate, citrate, fumurate, lactate, tartrate, maleate, maleate, succinate, or mixtures of two or more thereof.

The viscosity of the compositions of the present invention can be maintained at a desired level using a pharmaceutically acceptable thickening agent. For example, the viscosity may be at least 1000 cps; from about 1000 to about 10,000 cps; from about 2000 cps to about 6500 cps; or from about 2500 cps to about 5000 cps. Thickening agents that can be used in accordance with the present invention include, for example, methyl cellulose, xanthan gum, carboxymethyl cellulose, hydroxypropyl cellulose, carbomer, polyvinyl alcohol, alginates, acacia, chitosans, and mixtures of two or more thereof. The concentration of the thickening agent will depend upon the agent selected and the viscosity desired. Such agents can also be used in a powder formulation.

The nasally administrable compositions can also include a humectant to reduce or prevent drying of the mucus membrane and to prevent irritation thereof. Suitable humectants that can be used include, for example, sorbitol, mineral oil, vegetable oil and glycerol; soothing agents; membrane conditioners; sweeteners; and mixtures of two or more thereof. The concentration of the humectant will vary depending upon the agent selected. In one embodiment, the humectant can be present in the nasal delivery system in a concentration ranging from about 0.01% to about 20% by weight of the composition.

In other embodiments, the nasal delivery system can further comprise surfactants which enhance the absorption of the compound. Suitable surfactants include non-ionic, anionic and cationic surfactants. Exemplary surfactants include oleic acid, polyoxyethylene derivatives of fatty acids, partial esters of sorbitol anhydride, such as for example, Tweens (e.g., Tween 80, Tween 40, Tween 20), Spans (e.g., Span 40, Span 80, Span 20), polyoxyl 40 stearate, polyoxy ethylene 50 stearate, fusieates, bile salts, octoxynol, and mixtures of two or more thereof. Exemplary anionic surfactants include salts of long chain hydrocarbons (e.g., C6-30 or C 10-20) having one or more of the following functional groups: carboxylates; sulfonates; and sulfates. Salts of long chain hydrocarbons having sulfate functional groups are preferred, such as sodium cetostearyl sulfate, sodium dodecyl sulfate and sodium tetradecyl sulfate. One particularly preferred anionic surfactant is sodium lauryl sulfate (i.e., sodium dodecyl sulfate). The surfactants can be present in an amount from about 0.001% to about 50% by weight, or from about 0.001% to about 20% by weight.

The pharmaceutical compositions of the invention may further comprise an isotonicity agent, such as sodium chloride, dextrose, boric acid, sodium tartrate or other inorganic or organic solutes.

The nasal pharmaceutical compositions of the invention can optionally be used in combination with a pH adjusting agent. Exemplary pH adjusting agents include sulfuric acid, sodium hydroxide, hydrochloric acid, and the like.

To extend shelf life, preservatives can be added to the nasally administrable compositions. Suitable preservatives that can be used include benzyl alcohol, parabens, thimerosal, chlorobutanol, benzalkonium chloride, or mixtures of two or more thereof. Preferably benzalkonium chloride is used. Typically, the preservative will be present in a concentration of up to about 2% by weight. The exact concentration of the preservative, however, will vary depending upon the intended use and can be easily ascertained by one skilled in the art.

Other ingredients which extend shelf life can be added such as for example, antioxidants. Some examples of antioxidants include sodium metabisulfite, potassium metabisulfite, ascorbyl palmitate and the like. Typically, the antioxidant will be present in the compositions in a concentration of from about 0.001% up to about 5% by weight of the total composition.

The nasal delivery systems can be made following the processes described in, for example, U.S. Pat. Nos. 6,451,848, 6,436,950, and 5,874,450, and WO 00/00199, the disclosures of which are incorporated by reference herein in their entirety.

### EXAMPLES

The following examples are for purposes of illustration only and are non intended to limit the spirit or scope of the claims.

### Example 1

Anti-migraine agents are commonly evaluated using the carrageenan-induced thermal hyperalgesia model (Bingham et al, Experimental Neurology, 167:65-73 (2001); Daher et al, Life Sciences, 76:2349-2359 (2005)). The anti-migraine property of Compound A (i.e., 3-(2-cyanophenyl)-5-(2-pyridyl)-1-phenyl-1,2-dihydropyridin-2-one) on thermal hyperalgesia was evaluated in the following rat carrageenan-induced inflammatory pain model.

Male Wistar rats (5/group) were used for the experiment. Withdrawal latency to escape from heat noxious stimuli on both hind paws of the rats was measured using TAIL FLICK 7360 (Ugo Basile, Italy). 1% Carageenan was injected into the right hind paw footpat of the rats. 3-(2-cyanophenyl)-5-(2-pyridyl)-1-phenyl-1,2-dihydropyridin-2-one or vehicle was orally administered to the rats two hours after the carrageenan injection. The withdrawal latency from heat stimuli of both hind paws of rats was measured 1, 3 and 5 hours after drug administration.

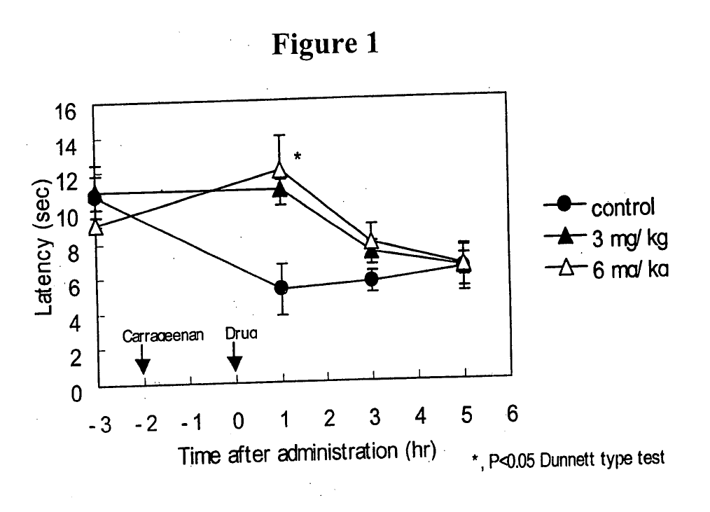

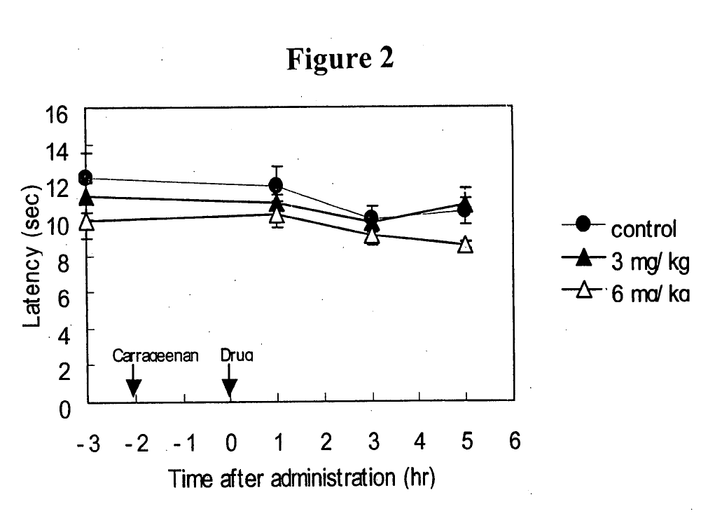

As shown in FIG. 1, the withdrawal latency of the carrageenan-injected right hind paws of the rats was shortened. As shown in FIG. 2, the withdrawal-latency of the left hind paw of the rats was not changed. The results show that carrageenan elicited inflammatory hyperalgesia in the injected hind paw and did not cause hyperalgesia in the contralateral hind paw of the rats. The effect of carrageenan was observed during all of the experiment period.

FIG. 1 also shows that 3-(2-cyanophenyl)-5-(2-pyridyl)-1-phenyl-1,2-dihydropyridin-2-one at 3 and 6 mg/kg inhibited induced hyperalgesia at 1 hour after administration, and the effect declined at 3 and 5 hours after administration. FIG. 2 also shows that 3-(2-cyanophenyl)-5-(2-pyridyl)-1-phenyl-1,2-dihydropyridin-2-one did not change the latency in the contralateral hind paw in the rats, so it is suggested that 3-(2-cyanophenyl)-5-(2-pyridyl)-1-phenyl-1,2-dihydropyridin-2-one did not affect motor function. These animal model results are predictive of the ability of 3-(2-cyanophenyl)-5-(2-pyridyl)-1-phenyl-1,2-dihydropyridin-2-one to treat migraines and headaches in humans.

### Example 2

A randomized double-blind, placebo-controlled, multi-center, parallel-group study is being conducted to evaluate the efficacy and safety of 3-(2-cyanophenyl)-5-(2-pyridyl)-1-phenyl-1,2-dihydropyridin-2-one hydrate in migraine prophylaxis and treatment.

The primary efficacy endpoint is to evaluate the efficacy of 23-(2-cyanophenyl)-5-(2-pyridyl)-1-phenyl-1,2-dihydropyridin-2-one hydrate in reducing migraines based on the change in the frequency of migraine periods per 28 days during the treatment phase compared to the baseline phase. A migraine period is defined as a migraine that starts, ends, or recurs within 24 hours. If the migraine persists for longer than 24 hours, it is considered a new migraine period.

Secondary objectives of the study are to evaluate the safety and tolerability of 23-(2-cyanophenyl)-5-(2-pyridyl)-1-phenyl-1,2-dihydropyridin-2-one hydrate in patients with migraines; to characterize the pharmacokinetics of 3-(2-cyanophenyl)-5-(2-pyridyl)-1-phenyl-1,2-dihydropyridin-2-one hydrate in the patient population; to determine the 3-(2-cyanophenyl)-5-(2-pyridyl)-1-phenyl-1,2-dihydropyridin-2-one hydrate plasma concentration-response relationship; and to assess the change in migraine severity. Secondary efficacy is determined based on a reduction in one or more of the following indices compared to Phase 1: (a) the proportion of patients experiencing at least a 50% reduction in a 28 day migraine period frequency; (b) change in migraine period frequency per 28 days during Phase 3; (c) change in migraine attack frequency; (d) change in the number of days requiring symptomatic rescue medication per 28 days; (e) total number of days with migraines per 28 days; (f) change in the severity of the migraine attack; (g) change in the duration of migraine attack; (h) change in the patients' Global Impression of Change; and (i) change in the Migraine Disability Assessment (MIDAS) questionnaire score.

For blood plasma concentration studies, the C_{MinSS}, C_{MaxSS} and AUC_{0-TauSS} are calculated by establishing a population pharmacokinetic model. Appropriate individual posterior estimates of pharmacokinetic parameters is also being determined. Inter-patient and inter-visit variability in these parameters is also being estimated. The study of the exposure-response (reduction in migraine frequency per 28 days) relationship is being explored using mixed-effect modeling.

Safety is being assessed by the incidence of adverse events, and changes in physical and neurological examinations, vital signs, clinical laboratory tests, weight, and electrocardiogram results.

The study is a 14 week, prospective, randomized, double-blind, placebo-controlled, multi-center, parallel-group study with a 4-week baseline phase and a 4-week single-blind placebo safety phase at the end of the study. The study has the following phases. Phase 1 is a 4-week baseline phase during which time a prospective ascertainment of migraine frequency is being assessed using the patients' migraine diary. Phase 2 is up to a 1-10 week titration phase starting at a dose of 0.5 to 2.0 mg/day which is being administered once in the evening. After 1-5 weeks the dose is being increased to 1.0 to 2.0 mg/day, and one to five weeks later the dose is being increased to 1.5 to 4.0 mg/day. Patients who are suffering intolerable adverse events are having the dose reduced one dose-step. During this phase, blood samples are being obtained for determination of plasma concentrations. During Phase 2, clinic visits are conducted every two weeks. Phase 3 is a 4 to 12 week maintenance phase where the dose is not being changed from the last dose that is administered during Phase 2. Blood plasma concentration samples are being obtained at each visit. During Phase 3, clinic visits are being conducted every four weeks. Phase 4 is a 4-week phase where all patients are receiving a placebo in a single-blind fashion. Patients are continuing to record the frequency and characteristics of their migraines. A final visit is scheduled about 4 weeks after Phase 3 and blood plasma concentration sample are being obtained at that time.

Patient eligibility is determined following the signing of the Informed Consent and during the Screening period. Screening assessments include a medical history, comprehensive physical and neurological examinations, vital signs, 12-lead ECG, clinical laboratory tests, urine drug screen, rival screen and administration of the MIDAS. During the Screening visit, eligible patients are instructed in the use of an electronic diary and asked to document the occurrence of migraines and other study-related information on a daily basis. At this time, patients enter into Phase 1.

Following successful completion of Phase 1 and verification that all of the inclusion and none of the exclusion criteria are met, patients are eligible to enter the study. Patients who experienced less than 4 or more than 12 migraines, failed to adequately complete the electronic diary, or failed to comply with trial procedures during Phase 1 is not being randomized.

Randomized patients are male and female, 18-65 years of age, and any race, with a history of migraines (with or without aura according to the Headache Classification Committee of the International Headache Society (HIS, 2004 guideline)) for at least 12 months with an onset before age 50, experiencing 4-12 migraine attacks per month during both the 3 months prior to Screening and Phase 1. The patients' body mass index (BMI) should be between 19 and 40 kg/m² inclusive at Screening.

180 patients are being enrolled in the study and are being randomized at a ratio of 1:1, with 90 patients in the placebo group and 90 patients receiving 3-(2-cyanophenyl)-5-(2-pyridyl)-1-phenyl-1,2-dihydropyridin-2-one hydrate. It is expected that 164 patients will complete the study. The study is being conducted at about 25 investigational sites in the United States.

Each of the patents, patent applications, and publications cited herein are incorporated by reference herein in their entirety.

It will be apparent to one skilled in the art that various modifications can be made to the invention without departing from the spirit or scope of the appended claims.

What is claimed is:

1. A method for treating a migraine in a patient in need thereof comprising administering a therapeutically effective amount of 3-(2-cyanophenyl)-5-(2-pyridyl)-1-phenyl-1,2-dihydropyridin-2-one, a pharmaceutically acceptable salt thereof, a hydrate thereof, or a hydrate of a pharmaceutically acceptable salt thereof.

2. The method of claim 1, wherein the therapeutically effective amount of 3-(2-cyanophenyl)-5-(2-pyridyl)-1-phenyl-1,2-dihydropyridin-2-one is from 30 [g to 10 grams.

3. The method of claim 1, wherein the therapeutically effective amount of 3-(2-cyanophenyl)-5-(2-pyridyl)-1-phenyl-1,2-dihydropyridin-2-one is from 100 μg to 100 mg.

4. The method of claim 1, wherein the migraine is with or without aura.

5. The method of claim 1, wherein the method for treating the migraine comprises a method for treating one or more migraine symptoms selected from the group consisting of vertigo, nausea, vomiting, fatigue, aura, photophobia, and phonophobia.

6. The method of claim 1, wherein the migraine is a classic migraines, a common migraine, a complicated migraine, a menstrual migraine, a premenstrual migraine, an ophthalmic migraine, an ophthalmoplegic migraine, a fulgurating migraine, a Harris' migraine, or a hemiplegic migraine.

7. The method of claim 1, wherein the method is for chronic treatment or acute treatment.

8. The method of claim 7, wherein the migraine is episodic or chronic.

9. A method for treating a headache in a patient in need thereof comprising administering a therapeutically effective amount of a compound of formula (I), a pharmaceutically acceptable salt thereof, a hydrate thereof, or a hydrate of a pharmaceutically acceptable salt thereof: 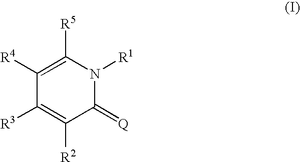
wherein:
Q is NH, O or S;
R¹, R², R³, R⁴ and R⁵ are each independently hydrogen, halogen, C₁₋₆alkyl or —X-A;
X is a single bond, an optionally substituted C₁₋₆ alkylene, an optionally substituted C₂₋₆ alkenylene, an optionally substituted C₂₋₆ alkynylene, —O—, —S—, —CO—, —SO—, —SO₂—, —N(R⁶)—, —N(R⁷)—CO—, —CO—N(R⁸)—, —N(R⁹)—CH₂—, —CH₂—N(R¹⁰)—, —CH₂—CO—, —CO—CH₂—, —N(R¹¹)—S(O)ₘ—, —S(O)ₙ—N(R²)—, —CH₂—S(O)ₚ—, —S(O)_{q}—CH₂—, —CH₂—O—, —O—CH₂—, —N(R¹³)'CO—N(R¹⁴)— or —N(R¹⁵)'CS—N(R¹⁶)—; p1 R⁶, R⁷, R⁸, R⁹, R¹⁰, R¹¹, R¹², R¹³, R¹⁴, R¹⁵ and R¹⁶ are each independently hydrogen C₁₋₆ alkyl or C₁₋₆ alkoxy;
m, n, p and q are each independently an integer of 0, 1 or 2;
A is an optionally substituted C₃₋₈ cycloalkyl, an optionally substituted C₃₋₈ cycloalkenyl, an optionally substituted 5- to 14-membered non-aromatic heterocyclic ring, an optionally substituted C₆₋₁₄ aromatic hydrocarbocyclic ring, or an optionally substituted 5- to 14-membered aromatic heterocyclic ring, provided that 3 groups among R¹, R², R³, R⁴ and R⁵ are —X-A; and that the residual 2 groups among R¹, R², R³, R⁴ and R⁵ are independently hydrogen, halogen, or C₁₋₆ alkyl.

10. The method of claim 9, wherein the headache is a primary headache or a secondary headache.

11. The method of claim 10, wherein the primary headache is a migraine, tension headache, cluster headache, paroxysmal hemicrania, short-lasting unilateral neuralgiform headache attack with conjunctival injection and tearing, trigeminal autonomic cephalalgia, stabbing headache, cough headache, exertional headache, headache associated with sexual activity, hypnic headache, thunderclap headache, hemicrania continua, or a new daily-persistent headache.

12. The method of claim 10, wherein the secondary headache is a headache attributed to a head and/or a neck trauma; a headache attributed to a cranial and/or a cervical vascular disorder; a headache attributed to a non-vascular intracranial disorder; a headache attributed to one or more drugs; a headache attributed to withdrawal from one or more drugs; a headache attributed to an infection; a headache attributed to a disturbance of homeostasis; a headache or attributed to a disorder of a facial structure and/or a cranial structure; or a headache attributed to a psychiatric disorder.

13. The method of claim 9, wherein the headache is a migraine.

14. The method of claim 13, wherein the migraine is with or without aura.

15. The method of claim 13, wherein the method for treating the migraine comprises a method for treating one or more migraine symptoms selected from the group consisting of vertigo, nausea, vomiting, fatigue, aura, photophobia, and phonophobia.

16. The method of claim 13, wherein the migraine is a classic migraines, a common migraine, a complicated migraine, a menstrual migraine, a premenstrual migraine, an ophthalmic migraine, an ophthalmoplegic migraine, a fulgurating migraine, a Harris' migraine, or a hemiplegic migraine.

17. The method of claim 13, wherein the method is for chronic treatment or acute treatment.

18. The method of claim 13, wherein the migraine is episodic or chronic.

19. The method of claim 9, wherein the therapeutically effective amount is from 30 μg to 10 grams.

20. The method of claim 9, further comprising administering a therapeutically effective amount of: (i) a cholinesterase inhibitor; (ii) an anti-migraine agent; or (iii) a cholinesterase inhibitor and an anti-migraine agent.

21. A method for treating a headache in a patient in need thereof comprising administering a therapeutically effective amount of: (i) a compound of Formula (III), a pharmaceutically acceptable salt thereof, a hydrate thereof, or a hydrate of a pharmaceutically acceptable salt thereof; and (ii) a compound of Formula (VI) or a pharmaceutically acceptable salt thereof,
wherein the compound of Formula (III) is: 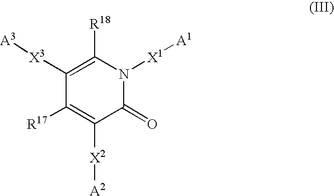
wherein
X¹, X² and X³ are each independently a single bond, an optionally substituted C₁₋₆ alkylene, an optionally substituted C₂₋₆ alkenylene, an optionally substituted C₂₋₆ alkynylene, —O—, —S—, —CO—, —SO—, —SO₂—, —N(R⁶)—, —N(R⁷)—CO—, —CO—N(R⁸)—, —N(R⁹)—CH₂—, —CH₂—N(R¹⁰)—, —CH₂—CO—, —CO—CH₂—, —N(R¹¹)—S(O)ₘ—, —S(O)ₙ—N(R¹²)—, —CH₂—S(O)ₚ—, —S(O)_{q}—CH₂—, —CH₂—O—, —O—CH₂—, —N(R¹³)—CO—N(R¹⁴)— or —N(R¹⁵)—CS—N(R¹⁶);
R⁶, R⁷, R⁸, R⁹, R¹⁰, R¹¹, R¹², R¹³, R¹⁴, R¹⁵ and R¹⁶ are each independently hydrogen C₁₋₆ alkyl, or C₁₋₆ alkoxy;
m, n, p and q are each independently an integer of 0, 1 or 2;
A¹, A²and A³ are each independently an optionally substituted C₃₋₈ cycloalkyl, an optionally substituted C₃₋₈ cycloalkenyl, an optionally substituted 5- to 14-membered non-aromatic heterocyclic ring, an optionally substituted C₆₋₁₄ aromatic hydrocarbocyclic ring, or an optionally substituted 5 to 14-membered aromatic heterocyclic ring; and
R¹⁷ and R¹⁸ are each independently hydrogen, halogen, or C₁₋₆ alkyl; and
wherein the compound of Formula (VI) is: 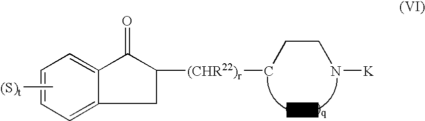
wherein
r is an integer of 1 to 10;
each R²² is independently hydrogen or methyl;
K is a phenalkyl or a phenalkyl having a substituent on the phenyl;
each S is independently hydrogen, C₁₋₆ lower alkyl, or C₁₋₆ lower alkoxy;
t is an integer of 1 to 4;
q is an integer of 1 to 3;
with the proviso that (S)t can be methylenedioxy or ethylenedioxy joined to two adjacent carbon atoms of the phenyl.

22. The method of claim 21, wherein the compound of Formula (III) is 3-(2-cyanophenyl)-5-(2-pyridyl)-1-phenyl-1,2-dihydropyridin-2-one, a pharmaceutically acceptable salt thereof, a hydrate thereof, or a hydrate of a pharmaceutically acceptable salt thereof.

23. The method of claim 21, wherein the compound of Formula (III) is administered in an amount of 30 μg to 10 grams.

24. The method of claim 21, wherein the compound of Formula (VI) is donepezil or a pharmaceutically acceptable salt thereof.

25. The method of claim 21, wherein the compound of Formula (VI) is administered in an amount from 1 mg to 50 mg.

26. The method of claim 21, wherein the headache is a migraine.

27. The method of claim 26, wherein the migraine is with or without aura.

28. The method of claim 26, wherein the method for treating the migraine comprises a method for treating one or more migraine symptoms selected from the group consisting of vertigo, nausea, vomiting, fatigue, aura, photophobia, and phonophobia.

29. The method of claim 26, wherein the migraine is a classic migraines, a common migraine, a complicated migraine, a menstrual migraine, a premenstrual migraine, an ophthalmic migraine, an ophthalmoplegic migraine, a fulgurating migraine, a Harris' migraine, or a hemiplegic migraine.

30. The method of claim 26, wherein the method is for chronic treatment or acute treatment.

31. The method of claim 26, wherein the migraine is episodic or chronic.

32. The method of claim 21, wherein the compound of Formula (III), the pharmaceutically acceptable salt thereof, the hydrate thereof, or the hydrate of the pharmaceutically acceptable salt thereof, and the compound of Formula (VI) or the pharmaceutically acceptable salt thereof, are administered separately to the patient.

33. The method of claim 21, wherein the compound of Formula (III), the pharmaceutically acceptable salt thereof, the hydrate thereof, or the hydrate of the pharmaceutically acceptable salt thereof; and the compound of Formula (VI) or the pharmaceutically acceptable salt thereof, are administered to the patient in the form of a pharmaceutical composition.

34. A pharmaceutical composition comprising a therapeutically effective amount of: (i) a compound of Formula (III), a pharmaceutically acceptable salt thereof, a hydrate thereof, or a hydrate of a pharmaceutically acceptable salt thereof; and (ii) a compound of Formula (VI) or a pharmaceutically acceptable salt thereof; wherein the compound of Formula (III) is: 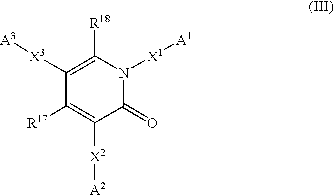
wherein
X¹, X² and X³ are each independently a single bond, an optionally substituted C₁₋₆ alkylene, an optionally substituted C₂₋₆ alkenylene, an optionally substituted C₂₋₆ alkynylene, —O—, —S—, —CO—, —SO—, —SO₂—, —N(R⁶)—, —N(R⁷)—CO—, —CO—N(R⁸)—, —N(R⁹)—CH₂—, —CH₂—N(R¹⁰)—, —CH₂—CO—, —CO—CH₂—, —N(R¹¹)—S(O)ₘ—, —S(O)ᵣ—N(R¹²)—, —CH₂—S(O)ₚ—, —S(O)_{q}—CH₂—, —CH₂—O—, —O—CH₂—, —N(R¹³)—CO—N(R¹⁴)— or —N(R¹⁵)—CS—N(R¹⁶);
R⁶, R⁷, R⁸, R⁹, R¹⁰, R¹¹, R¹², R¹³, R¹⁴, R¹⁵ and R¹⁶ are each independently hydrogen, C₁₋₆ alkyl, or C₁₋₆ alkoxy;
m, n, p and q are each independently an integer of 0, 1 or 2;
A¹, A² and A³ are each independently an optionally substituted C₃₋₈ cycloalkyl, an optionally substituted C₃₋₈ cycloalkenyl, an optionally substituted 5- to 14-membered non-aromatic heterocyclic ring, an optionally substituted C₆₋₁₄ aromatic hydrocarbocyclic ring, or an optionally substituted 5 to 14-membered aromatic heterocyclic ring; and
R¹⁷ and R¹⁸ are each independently hydrogen, halogen, or C₁₋₆ alkyl; and wherein the compound of Formula (VI) is: 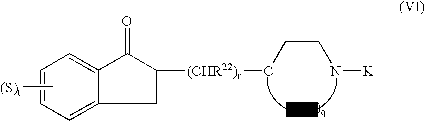
wherein
r is an integer of 1 to 10;
each R²² is independently hydrogen or methyl;
K is a phenalkyl or a phenalkyl having a substituent on the phenyl;
each S is independently hydrogen, C₁₋₆ lower alkyl, or C₁₋₆ lower alkoxy;
t is an integer of 1 to 4;
q is an integer of 1 to 3;
with the proviso that (S)t can be methylenedioxy or ethylenedioxy joined to two adjacent carbon atoms of the phenyl.

35. The composition of claim 34, wherein the compound of Formula (III) is 3-(2-cyanophenyl)-5-(2-pyridyl)-1-phenyl-1,2-dihydropyridin-2-one, a pharmaceutically acceptable salt thereof, a hydrate thereof, or a hydrate of a pharmaceutically acceptable salt thereof.

36. The composition of claim 34, wherein the compound of Formula (III) is present in an amount of 30 μg to 10 grams.

37. The composition of claim 34, wherein the compound of Formula (VI) is donepezil or a pharmaceutically acceptable salt thereof.

38. The composition of claim 34, wherein the compound of Formula (VI) is present in an amount from 1 mg to 50 mg.
